(19) United States
(12) Patent Application Publication
(10) Pub. No.: US 2024/0189037 A1
(43) Pub. Date: Jun. 13, 2024

(54) PATIENT-SPECIFIC IMPLANT DESIGN AND MANUFACTURING SYSTEM WITH A REGULATORY AND REIMBURSEMENT MANAGER

(71) Applicant: Carlsmed, Inc., Carlsbad, CA (US)

(72) Inventor: Niall Patrick Casey, Carlsbad, CA (US)

(21) Appl. No.: 18/537,600

(22) Filed: Dec. 12, 2023

Related U.S. Application Data
(60) Provisional application No. 63/387,009, filed on Dec. 12, 2022.

(51) Int. Cl. A61B 34/10; A61B 34/00; G16H 10/60; G16H 40/20

(52) U.S. Cl. CPC A61B 34/10; A61B 34/25; G16H 10/60; G16H 40/20; A61B 2034/104; A61B 2034/105; A61B 2034/108; A61B 2034/256

(57) ABSTRACT

Systems and methods for designing and implementing patient-specific surgical procedures and/or medical devices are disclosed. In some embodiments, a method includes receiving a patient data set of a patient. The patient data set is compared to a plurality of reference patient data sets, wherein each of the plurality of reference patient data sets is associated with a corresponding reference patient. A subset of the plurality of reference patient data sets is selected based, at least partly, on similarity to the patient data set and treatment outcome of the corresponding reference patient. Based on the selected subset, at least one surgical procedure or medical device design for treating the patient is generated.

### CROSS-REFERENCE TO RELATED APPLICATION

This application claims priority to U.S. Provisional Application No. 63/387,009, filed Dec. 12, 2022, which is hereby incorporated by reference in its entirety.

### TECHNICAL FIELD

The present disclosure is generally related to designing, manufacturing, and implementing medical care, and more particularly to systems and methods for managing regulatory and reimbursements for patient-specific surgical procedures and/or medical devices.

### BACKGROUND

Numerous types of data associated with patient treatments and surgical interventions are available. To determine treatment protocols for a patient, physicians often rely on a subset of patient data available via the patient's medical record and historical outcome data. However, the amount of patient data and historical data may be limited, and the available data may not be correlated or relevant to the particular patient to be treated. Additionally, although digital data collection and processing power have improved, technologies using collected data to determine optimal treatment protocols have lagged. For example, conventional technologies in the field of orthopedics may lack the capability to draw upon large data sets to generate and optimize patient-specific treatments (e.g., surgical interventions and/or implant designs) to achieve favorable treatment outcomes. Unfortunately, it may be difficult to plan surgical procedures that meet economic or financial requirements (e.g., reimbursement requirements) while achieving such favorable treatment outcomes. Healthcare reimbursement describes the payment that a hospital, healthcare provider, diagnostic facility, or other healthcare providers receive for providing a medical service.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a network connection diagram illustrating a system for providing patient-specific medical care, according to an embodiment.

FIG. 2 illustrates a computing device suitable for use in connection with the system of FIG. 1, according to an embodiment.

FIG. 3 is a flow diagram illustrating a method for providing patient-specific medical care, according to an embodiment.

FIGS. 4A-4C illustrate exemplary data sets that may be used and/or generated in connection with the methods described herein, according to an embodiment. FIG. 4A illustrates a patient data set. FIG. 4B illustrates a plurality of reference patient data sets.

FIG. 4C illustrates similarity scores and outcome scores for the reference patient data sets of FIG. 4B.

FIG. 5 is a flow diagram illustrating another method for providing patient-specific medical care, according to an embodiment.

FIGS. 6A-6B are flow diagrams illustrating methods for providing reimbursable patient-specific medical care, according to an embodiment.

FIGS. 7A-7D illustrates an exemplary patient data set that may be used and/or generated in connection with the methods described herein, according to an embodiment.

FIGS. 8A and 8B illustrate an exemplary virtual model of a patient's spine that may be used and/or generated in connection with the methods described herein, according to an embodiment.

FIGS. 9A-1-9B-2 illustrate an exemplary virtual model of a patient's spine in a pre-operative anatomical configuration and a corrected anatomical configuration. More specifically, FIGS. 9A-1 and 9A-2 illustrates the pre-operative anatomical configuration of the patient, FIGS. 9B-1 and 9B-2 illustrates the corrected anatomical configuration.

FIG. 10A illustrates an exemplary interactive surgical plan for a patient-specific surgical procedure, according to an embodiment.

FIG. 10B illustrates a code report for a treatment plan or a patient, according to an embodiment.

FIG. 11 illustrates an exemplary surgical plan report detailing the surgical plan for surgeon review and that may be used and/or generated in connection with the methods described herein, according to an embodiment.

FIGS. 12A and 12B illustrate an exemplary patient-specific implant that can be used and/or generated in connection with the methods described herein, according to an embodiment.

FIG. 13 illustrates a segment of a patient's spine after several patient-specific implants have been implanted therein.

### DETAILED DESCRIPTION

The present technology is directed to systems and methods managing schemes (e.g., regulatory schemes, reimbursement schemes, etc.) for personalized medicine. The present technology can display, via a display on a device, a patient-specific interactive surgical plan generated by a surgical planning platform based on one or more images of a patient. The patient-specific interactive surgical plan can include a user input element for modifying and/or approving the interactive surgical plan. In some cases, the interactive surgical plan includes a viewable planned post-operative pathology for the patient. The system can obtain reimbursement information (amounts, availability, etc.) for one or more patient-specific implants based on pathology data of the patient in the interactive surgical plan and associate at least one medical reimbursement code to the one or more patient-specific implants based on the reimbursement information. The system can receive, via the interactive surgical plan, approval of or modification to the planned pathology. In some embodiments, the surgical planning platform is configured to design the one or more patient-specific implants for achieving the approved planned post-operative pathology and for medical reimbursement under the at least one medical reimbursement code.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. As used herein and in the appended claims, the singular forms “a,” “an,” and “the” include plural references unless the context clearly dictates otherwise.

Although the disclosure herein primarily describes systems and methods for treatment planning in the context of orthopedic surgery, the technology may be applied equally to medical treatment and devices in other fields (e.g., other types of surgical practice). Additionally, although many embodiments herein describe systems and methods with respect to implanted devices, the technology may be applied equally to other types of medical devices (e.g., non-implanted devices).

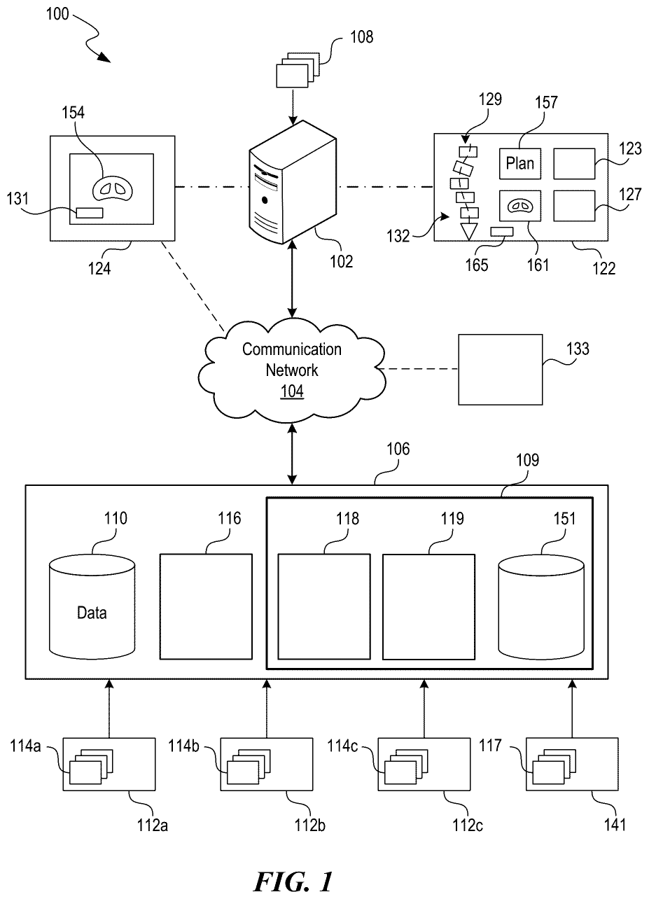

FIG. 1 is a network connection diagram illustrating a computing system 100 for patient-specific medical care, according to an embodiment. As described in further detail herein, the system 100 is configured to generate a medical treatment plan based on patient data, patient insurance information, regulatory requirements, reimbursement information, or the like. The system 100 can design treatments covered by, for example, the patient's insurance plan to manage patient costs, manage reimbursements/payment by third-party payors, manage regulatory compliance, and/or manufacture patient-specific devices. The system 100 can synchronize steps to optimize patient outcomes while meeting regulatory/financial criteria. The system 100 can manufacture Food and Drug Administration (FDA)-approved devices, such as surgical kits, implants, instruments, etc. This reduces the risk of patient-specific items not resulting in reimbursement and/or predicted targeted outcomes(s).

The system 100 can generate regulatory compliant and reimbursable treatment plans (“RCR treatment plans”) designed for regulatory compliance while meeting reimbursable requirements. For example, the RCR treatment plans can include pre-operative metrics (e.g., pre-operative measurements), predicted or corrected pathologies, regulatory compliance verification, payment or reimbursement information, insurance information, billing information, or the like and can identify and link relationships between regulatory compliance, payment/reimbursement, and treatment for user review. This allows a user to visually perform a comprehensive review. For example, a physician can view the RCR treatment plan to approve a predicted post-operative pathology, a healthcare provider can review the RCR treatment plan to verify payment/reimbursement (e.g., preoperative verification prior to starting a surgical intervention), and a manufacturer can manufacture governmental-approved surgical items. The surgical items can be analyzed to confirm that each item complies with regulatory requirements. The RCR treatment plan can also be used to generate post-operative billing submissions, such as third-party payor submissions (e.g., payment or reimbursement submissions). Accordingly, the RCR treatment plan can be used at different stages of pre-operative activities, intra-operative activities, and/or post-operative activities.

The RCR treatment plans can be designed to treat orthopedic or spinal disease or disorder, such as trauma (e.g., fractures), cancer, deformity, degeneration, pain (e.g., back pain, leg pain), irregular spinal curvature (e.g., scoliosis, lordosis, kyphosis), irregular spinal displacement (e.g., spondylolisthesis, lateral displacement, axial displacement), osteoarthritis, lumbar degenerative disc disease, cervical degenerative disc disease, lumbar spinal stenosis, or cervical spinal stenosis, or a combination thereof. The medical treatment plan can include regulatory information, reimbursement information, surgical plan, technology recommendations (e.g., device and/or instrument recommendations), and/or medical device designs. For example, the medical treatment plan can include at least one treatment procedure (e.g., a surgical procedure or intervention) and/or at least one medical device (e.g., an implanted medical device (also referred to herein as an “implant” or “implanted device”) or implant delivery instrument). Implants can be implanted manually, robotically, under navigation, or other selected techniques. Planned interventions can include selected interbody levels of the spine, interbody approaches, fixation levels, osteotomy(ies), etc.

The RCR treatment plan can be part of or include a medical treatment plan that is customized for a particular patient or group of patients, also referred to herein as a “patient-specific” or “personalized” treatment plan. The patient-specific treatment plan can include at least one patient-specific surgical procedure and/or at least one patient-specific medical device that are designed and/or optimized for the patient's particular characteristics (e.g., condition, anatomy, pathology, condition, medical history). For example, the patient-specific medical device can be designed and manufactured specifically for the particular patient, rather than being an off-the-shelf device. However, it shall be appreciated that a patient-specific treatment plan can also include aspects that are not customized for the particular patient. For example, a patient-specific or personalized surgical procedure can include one or more instructions, portions, steps, etc. that are non-patient-specific. Likewise, a patient-specific or personalized medical device can include one or more components that are non-patient-specific, and/or can be used with an instrument or tool that is non-patient-specific. Personalized implant designs can be used to manufacture or select patient-specific technologies, including medical devices, instruments, and/or surgical kits. For example, a personalized surgical kit can include one or more patient-specific devices, patient-specific instruments, non-patient-specific technology (e.g., standard instruments, devices, etc.), instructions for use, patient-specific treatment plan information, or a combination thereof.

The system 100 can identify diagnoses for a patient (e.g., primary or second diagnoses) that meet at least one of a diagnosis code, a procedure code, and/or a supply item code. The system 100 can submit a payment request for the one or more patient-specific implants under the at least one of the diagnosis code(s), the supply item code(s), or the procedure code(s). In some embodiments, the payment request can be submitted via a payment portal, website, or other payment submission system. The system 100 can display an RCR treatment plan 132 based on constraints (e.g., governmental-approved design constraints) from a database 151 to generate a corrected pathology 129. The system 100 can design, using the design constraint(s), patient-specific implant(s) 161 configured to achieve the corrected pathology while meeting requirements for coverage and medical reimbursement. For example, the system 100 can plan medical services (e.g., surgical procedures, therapies, etc.) that meet economic or financial requirements (e.g., reimbursement requirements) or other parameters while achieving favorable treatment outcomes. The system 100 can identify and/or determine healthcare reimbursements, third party payments, or other payments that a hospital, healthcare provider, diagnostic facility, or other healthcare providers may receive for providing the medical service. A reimbursement machine-learning model of the system 100 can be trained using selected reference patient data sets to generate the corrected patient pathology 129 that meets one or more coverage or reimbursement parameters, economic parameters (e.g., level of profitability, threshold profitability, etc.), financial parameters, regulatory parameters, clinical parameters, or other parameters.

The corrected pathology 129 can be represented by one or more virtual models representing the patient's anatomy and a user can manipulate the virtual models to generate one or more surgical plans that qualifies for payment under at least one code stored by the database 151. The server 106 can manipulate the virtual model to generate additional surgical plans that qualifies for reimbursement under code(s) in the database 151. The display 122 can display a comparison of multiple plans with associated codes, which can be medical codes of insurers (e.g., private insurers, public insurers, etc.), government agencies, healthcare provides, etc. The codes can identify, for example, procedure(s) performed, diagnosis or diagnoses, item(s) (e.g., devices, supplies, and/or equipment) acquired for the patient, or the like. The display 122 can display additional surgical plans, regulatory data, reimbursements data, and other information can be compared and displayed. The manufacturing system 124 can then manufacture regulatory approved or unapproved items 154 designed for coverage/reimbursement.

The system 100 includes a client computing device 102, which can be a user device, such as a smart phone, mobile device, laptop, desktop, personal computer, tablet, phablet, or other such devices known in the art. As discussed further herein, the client computing device 102 can include one or more processors, and memory storing instructions executable by the one or more processors to perform the methods described herein. The client computing device 102 can be associated with a healthcare provider that is treating the patient. Although FIG. 1 illustrates a single client computing device 102, in alternative embodiments, the client computing device 102 can instead be implemented as a client computing system encompassing a plurality of computing devices, such that the operations described herein with respect to the client computing device 102 can instead be performed by the computing system and/or the plurality of computing devices.

The client computing device 102 is configured to receive a patient data set 108 associated with a patient to be treated. The patient data set 108 can include data representative of the patient's condition, anatomy, pathology, medical history, preferences, and/or any other information or parameters relevant to the patient. For example, the patient data set 108 can include medical history, surgical intervention data, treatment outcome data, progress data (e.g., physician notes), patient feedback (e.g., feedback acquired using quality of life questionnaires, surveys), clinical data, provider information (e.g., physician, hospital, surgical team), patient information (e.g., demographics, sex, age, height, weight, type of pathology, occupation, activity level, tissue information, health rating, comorbidities, health related quality of life (HRQL)), vital signs, diagnostic results, medication information, allergies, image data (e.g., camera images, Magnetic Resonance Imaging (MRI) images, ultrasound images, Computerized Aided Tomography (CAT) scan images, Positron Emission Tomography (PET) images, X-Ray images), diagnostic equipment information (e.g., manufacturer, model number, specifications, user-selected settings/configurations, etc.), or the like. In some embodiments, the patient data set 108 includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine.

The client computing device 102 is operably connected via a communication network 104 to a server 106, thus allowing for data transfer between the client computing device 102 and the server 106. The communication network 104 may be a wired and/or a wireless network. The communication network 104, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 106, which may also be referred to as a “treatment assistance network” or “prescriptive analytics network,” can include one or more computing devices and/or systems. As discussed further herein, the server 106 can include one or more processors, and memory storing instructions executable by the one or more processors to perform the methods described herein. In some embodiments, the server 106 is implemented as a distributed “cloud” computing system or facility across any suitable combination of hardware and/or virtual computing resources.

The client computing device 102 and server 106 can individually or collectively perform the various methods described herein for providing patient-specific medical care. For example, some or all of the steps of the methods described herein can be performed by the client computing device 102 alone, the server 106 alone, or a combination of the client computing device 102 and the server 106. Thus, although certain operations are described herein with respect to the server 106, it shall be appreciated that these operations can also be performed by the client computing device 102, and vice-versa.

The server 106 includes at least one database 110 configured to store reference data useful for the treatment planning methods described herein. The reference data can include historical and/or clinical data from the same or other patients, data collected from prior surgeries and/or other treatments of patients by the same or other healthcare providers, data relating to medical device designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like.

In some embodiments, the database 110 includes a plurality of reference patient data sets, each patient reference data set associated with a corresponding reference patient. For example, the reference patient can be a patient that previously received treatment or is currently receiving treatment. Each reference patient data set can include data representative of the corresponding reference patient's condition, anatomy, pathology, medical history, disease progression, preferences, and/or any other information or parameters relevant to the reference patient, such as any of the data described herein with respect to the patient data set 108. In some embodiments, the reference patient data set includes pre-operative data, intra-operative data, and/or post-operative data. For example, a reference patient data set can include data representing one or more of patient ID, age, gender, BMI, lumbar lordosis, Cobb angle(s), pelvic incidence, disc height, segment flexibility, bone quality, rotational displacement, and/or treatment level of the spine. As another example, a reference patient data set can include treatment data regarding at least one treatment procedure performed on the reference patient, such as descriptions of surgical procedures or interventions (e.g., surgical approaches, bony resections, surgical maneuvers, corrective maneuvers, placement of implants or other devices). In some embodiments, the treatment data includes medical device design data for at least one medical device used to treat the reference patient, such as physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties). In yet another example, a reference patient data set can include outcome data representing an outcome of the treatment of the reference patient, such as corrected anatomical metrics, presence of fusion, HRQL, activity level, return to work, complications, recovery times, efficacy, mortality, and/or follow-up surgeries.

In some embodiments, the server 106 receives at least some of the reference patient data sets from a plurality of healthcare provider computing systems (e.g., systems 112a-112c, collectively 112). The server 106 can be connected to the healthcare provider computing systems 112 via one or more communication networks (not shown). Each healthcare provider computing system 112 can be associated with a corresponding healthcare provider (e.g., physician, surgeon, medical clinic, hospital, healthcare network, etc.). Each healthcare provider computing system 112 can include at least one reference patient data set (e.g., reference patient data sets 114a-114c, collectively 114) associated with reference patients treated by the corresponding healthcare provider. The reference patient data sets 114 can include, for example, electronic medical records, electronic health records, biomedical data sets, biomechanical data sets, mobility data sets, pain data sets, reimbursement information (e.g., reimbursement history, reimbursement codes, etc.), payment information, insurance information, insurer information, etc. The reference patient data sets 114 can be received by the server 106 from the healthcare provider computing systems 112 and can be reformatted into different formats for storage in the database 110. Optionally, the reference patient data sets 114 can be processed (e.g., cleaned) to ensure that the represented patient parameters are likely to be useful in the treatment planning methods described herein.

The server 106 can receive at least some information from a regulatory or reimbursement system or provider 141 (“regulatory or reimbursement system 141”). For example, the server 106 can be connected to the system 141 via one or more communication networks (not shown). The system 141 can include one or more outcome data databases, reimbursement databases, regulatory databases, or the like. In some embodiments, the system 141 can include or be an agency database, such as an FDA database, Medicare database, Medicaid database, or the like. The server 106 can request and retrieve data sets 117 from the system 141.

As described in further detail herein, the server 106 can be configured with one or more algorithms that generate regulatory data, billing data (e.g., payment data, reimbursement data, etc.), and/or patient-specific treatment plan data (e.g., treatment procedures, medical devices, etc.) based on the reference data. In some embodiments, the patient-specific data is generated based on correlations between the patient data set 108 and the reference data. Optionally, the server 106 can predict outcomes, including recovery times, efficacy based on clinical end points, likelihood of success, predicted mortality, predicted related follow-up surgeries, or the like. In some embodiments, the server 106 can continuously or periodically analyze patient data (including patient data obtained during the patient stay) to determine near real-time or real-time risk scores, mortality prediction, etc.

In some embodiments, the server 106 includes one or more modules for performing one or more steps of the patient-specific treatment planning methods described herein. For example, in the depicted embodiment, the server 106 includes a data analysis module 116 and a surgical planning and reimbursement platform 109 (“SPR platform 109”). The SPR platform 109 includes a treatment planning module 118, a regulatory and reimbursement manager 119, and a database 151. In alternative embodiments, one or more of these modules may be combined with each other, or may be omitted. Thus, although certain operations are described herein with respect to a particular module or modules, this is not intended to be limiting, and such operations can be performed by a different module or modules in alternative embodiments. For example, the SPR platform 109 can be incorporated into the data analysis module 116. In other embodiments, the modules of the system 100 can be combined with modules of other systems. For example, the SPR platform 109 can be part of or incorporated into a healthcare system 133 and can manage billing, tracking reimbursement submissions, and/or manage payments.

The data analysis module 116 is configured with one or more algorithms for identifying a subset of reference data from the database 110 that is likely to be useful in developing a patient-specific treatment plan. For example, the data analysis module 116 can compare patient-specific data (e.g., the patient data set 108 received from the client computing device 102) to the reference data from the database 110 (e.g., the reference patient data sets) to identify similar data (e.g., one or more similar patient data sets in the reference patient data sets). The comparison can be based on one or more parameters, such as age, gender, BMI, lumbar lordosis, pelvic incidence, and/or treatment levels. The parameter(s) can be used to calculate a similarity score for each reference patient. The similarity score can represent a statistical correlation between the patient data set 108 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference is below a threshold can be considered to be similar patients.

The data analysis module 116 can further be configured with one or more algorithms to select a subset of the reference patient data sets, e.g., based on similarity to the patient data set 108 and/or treatment outcome of the corresponding reference patient. For example, the data analysis module 116 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, presence of fusion, HRQL, activity level, complications, recovery times, efficacy, mortality, or follow-up surgeries. As described in further detail below, in some embodiments, the data analysis module 116 calculates an outcome score by assigning values to each outcome parameter. A patient can be considered to have a favorable outcome if the outcome score is above, below, or at a specified threshold value.

In some embodiments, the data analysis module 116 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 116 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular health-care provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and other data sets can be utilized. By way of example, a patient-specific treatment plan to perform a battlefield surgery can be based on reference patient data from similar battlefield surgeries and/or data sets associated with battlefield surgeries. In another example, the patient-specific treatment plan can be generated based on available robotic surgical systems. The reference patient data sets can be selected based on patients that have been operated on using comparable robotic surgical systems under similar conditions (e.g., size and capabilities of surgical teams, hospital resources, etc.).

The SPR platform 109 can include the treatment planning module 118, the regulatory reimbursement manager 119, and the database 151. The treatment planning module 118 is configured with one or more algorithms to generate at least one treatment plan (e.g., pre-operative plans, surgical plans, post-operative plans etc.) based on the output from the data analysis module 116. In some embodiments, the treatment planning module 118 is configured to develop and/or implement at least one predictive model for generating the patient-specific treatment plan, also known as a “prescriptive model.” The predictive model(s) can be developed using clinical knowledge, statistics, machine learning, AI, neural networks, or the like. In some embodiments, the output from the data analysis module 116 is analyzed (e.g., using statistics, machine learning, neural networks, AI) to identify correlations between data sets, patient parameters, healthcare provider parameters, healthcare resource parameters, treatment procedures, medical device designs, and/or treatment outcomes. These correlations can be used to develop at least one predictive model that predicts the likelihood that a treatment plan will produce a favorable outcome for the particular patient. The predictive model(s) can be validated, e.g., by inputting data into the model(s) and comparing the output of the model to the expected output.

In some embodiments, the treatment planning module 118 is configured to generate the treatment plan based on previous treatment data from reference patients. For example, the treatment planning module 118 can receive a selected subset of reference patient data sets and/or similar patient data sets from the data analysis module 116, and determine or identify treatment data from the selected subset. The treatment data can include, for example, treatment procedure data (e.g., surgical procedure or intervention data) and/or medical device design data (e.g. implant design data) that are associated with favorable or desired treatment outcomes for the corresponding patient. The treatment planning module 118 can analyze the treatment procedure data and/or medical device design data to determine an optimal treatment protocol for the patient to be treated. For example, the treatment procedures and/or medical device designs can be assigned values and aggregated to produce a treatment score. The patient-specific treatment plan can be determined by selecting treatment plan(s) based on the score (e.g., higher or highest score; lower or lowest score; score that is above, below, or at a specified threshold value). The personalized patient-specific treatment plan can be based on, at least in part, the patient-specific technologies or patient-specific selected technology.

Alternatively or in combination, the treatment planning module 118 can generate the treatment plan based on correlations between data sets. For example, the treatment planning module 118 can correlate treatment procedure data and/or medical device design data from similar patients with favorable outcomes (e.g., as identified by the data analysis module 116). Correlation analysis can include transforming correlation coefficient values to values or scores. The values/scores can be aggregated, filtered, or otherwise analyzed to determine one or more statistical significances. These correlations can be used to determine treatment procedure(s) and/or medical device design(s) that are optimal or likely to produce a favorable outcome for the patient to be treated.

Alternatively or in combination, the treatment planning module 118 can generate the treatment plan using one or more AI techniques. AI techniques can be used to develop computing systems capable of simulating aspects of human intelligence, e.g., learning, reasoning, planning, problem solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the treatment planning module 118 generates the treatment plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 110, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training data set can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate a treatment plan, the patient data set 108 can be input into the trained machine learning model(s). Additional data, such as the selected subset of reference patient data sets and/or similar patient data sets, and/or treatment data from the selected subset, can also be input into the trained machine learning model(s). The trained machine learning model(s) can then calculate whether various candidate treatment procedures and/or medical device designs are likely to produce a favorable outcome for the patient, meet one or more parameters (e.g., coverage parameters, reimbursement parameters, regulatory parameters, or the like). Based on these calculations, the trained machine learning model(s) can select at least one treatment plan for the patient. In embodiments where multiple trained machine learning models are used, the models can be run sequentially or concurrently to compare outcomes and can be periodically updated using training data sets. The treatment planning module 118 can use one or more of the machine learning models based the model's predicted accuracy score.

The patient-specific treatment plan generated by the treatment planning module 118 can include at least one patient-specific treatment procedure (e.g., a surgical procedure or intervention) and/or at least one patient-specific medical device (e.g., an implant or implant delivery instrument). A patient-specific treatment plan can include an entire surgical procedure or portions thereof. Additionally, one or more patient-specific medical devices can be specifically selected or designed for the corresponding surgical procedure, thus allowing for the various components of the patient-specific technology to be used in combination to treat the patient.

In some embodiments, the patient-specific treatment procedure includes an orthopedic surgery procedure, such as spinal surgery, hip surgery, knee surgery, jaw surgery, hand surgery, shoulder surgery, elbow surgery, total joint reconstruction (arthroplasty), skull reconstruction, foot surgery, or ankle surgery. Spinal surgery can include spinal fusion surgery, such as posterior lumbar interbody fusion (PLIF), cervical fusion, anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). In some embodiments, the patient-specific treatment procedure includes descriptions of and/or instructions for performing one or more aspects of a patient-specific surgical procedure. For example, the patient-specific surgical procedure can include one or more of a surgical approach, a corrective maneuver, a bony resection, or implant placement.

In some embodiments, the patient-specific medical device design includes a design for an orthopedic implant and/or a design for an instrument for delivering an orthopedic implant. Examples of such implants include, but are not limited to, screws (e.g., bone screws, spinal screws, pedicle screws, facet screws), interbody implant devices (e.g., intervertebral implants), cages, plates, rods, disks, fusion devices, spacers, rods, expandable devices, stents, brackets, ties, scaffolds, fixation device, anchors, nuts, bolts, rivets, connectors, tethers, fasteners, joint replacements, hip implants, or the like. Examples of instruments include, but are not limited to, screw guides, cannulas, ports, catheters, insertion tools, or the like.

A patient-specific medical device design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of a corresponding medical device. For example, a design for an orthopedic implant can include implant shape, size, material, and/or effective stiffness (e.g., lattice density, number of struts, location of struts, etc.). In some embodiments, the generated patient-specific medical device design is a design for an entire device. Alternatively, the generated design can be for one or more components of a device, rather than the entire device.

In some embodiments, the design is for one or more patient-specific device components that can be used with standard, off-the-shelf components. For example, in a spinal surgery, a pedicle screw kit can include both standard components and patient-specific customized components. In some embodiments, the generated design is for a patient-specific medical device that can be used with a standard, off-the-shelf delivery instrument. For example, the implants (e.g., screws, screw holders, rods) can be designed and manufactured for the patient, while the instruments for delivering the implants can be standard instruments. This approach allows the components that are implanted to be designed and manufactured based on the patient's anatomy and/or surgeon's preferences to enhance treatment. The patient-specific devices described herein are expected to improve delivery into the patient's body, placement at the treatment site, and/or interaction with the patient's anatomy.

In embodiments where the patient-specific treatment plan includes a surgical procedure to implant a medical device, the treatment planning module 118 can also store various types of implant surgery information, such as implant parameters (e.g., types, dimensions), availability of implants, aspects of a pre-operative plan (e.g., initial implant configuration, detection and measurement of the patient's anatomy, etc.), FDA requirements for implants (e.g., specific implant parameters and/or characteristics for compliance with FDA regulations), or the like. In some embodiments, the treatment planning module 118 can convert the implant surgery information into formats useable for machine-learning based models and algorithms. For example, the implant surgery information can be tagged with particular identifiers for formulas or can be converted into numerical representations suitable for supplying to the trained machine learning model(s). The treatment planning module 118 can also store information regarding the patient's anatomy, such as two- or three-dimensional images or models of the anatomy, and/or information regarding the biology, geometry, and/or mechanical properties of the anatomy. The anatomy information can be used to inform implant design and/or placement.

The treatment plan(s) generated by the treatment planning module 118 can be transmitted via the communication network 104 to the client computing device 102 for output to a user (e.g., clinician, surgeon, healthcare provider, patient). In some embodiments, the client computing device 102 includes or is operably coupled to a display 122 for outputting the treatment plan(s). The display 122 can include a graphical user interface (GUI) for visually depicting various aspects of the treatment plan(s). For example, the display 122 can show various aspects of a surgical procedure to be performed on the patient, such as the surgical approach, treatment levels, corrective maneuvers, tissue resection, and/or implant placement. To facilitate visualization, a virtual model of the surgical procedure can be displayed. As another example, the display 122 can show a design for a medical device to be implanted in the patient, such as a two- or three-dimensional model of the device design. The display 122 can also show patient information, such as two- or three-dimensional images or models of the patient's anatomy where the surgical procedure is to be performed and/or where the device is to be implanted. The client computing device 102 can further include one or more user input devices (not shown) allowing the user to modify, select, approve, and/or reject the displayed treatment plan(s).

The regulatory and reimbursement manager 119 can analyze and manage regulatory requirements, regulatory compliance of treatment plans, reimbursement data (e.g., reimbursement requirements, coding, etc.) and other information. The database 151 can search for, retrieve, and store data from the regulatory and reimbursement from systems 141 or other systems. For example, the server 106 can be trained to generate new treatment plans, and the database 151 can identify candidate reimbursements for the new treatments. The database 151 can then retrieve the candidate reimbursement data sets from the system 141. The regulatory and reimbursement manager 119 can analyze and develop a reimbursement or payment plan.

Additionally, the treatment planning module 116 and/or the module 109 can correlate information between new treatments and regulatory and reimbursement data to generate treatment plans that meet target criteria, such as FDA-approved implants, payment/reimbursement thresholds, treatment outcomes, combinations thereof, or the like. This information can be provided to the healthcare system 133, user device 102, display 122, or other components of the system 100. This allows multiple users or entities to coordinate activities to provide comprehensive healthcare based on regulatory schemes, reimbursement/payment schemes, insurance schemes, or the like.

In some embodiments, the medical device design(s) generated by the server 106 can be transmitted from the client computing device 102 and/or server 106 to a manufacturing system 124 for manufacturing a corresponding medical device. The manufacturing system 124 can be located on site or off site. On-site manufacturing can reduce the number of sessions with a patient and/or the time to be able to perform the surgery whereas off-site manufacturing can be useful make the complex devices. Off-site manufacturing facilities can have specialized manufacturing equipment. In some embodiments, more complicated device components can be manufactured off site, while simpler device components can be manufactured on site.

Various types of manufacturing systems are suitable for use in accordance with the embodiments herein. Manufacturing can be achieved using human design, machine design, a combination of human and machine design, or other design techniques. For example, the manufacturing system 124 can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system 124 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 124 can manufacture one or more patient-specific medical devices based on fabrication instructions or data (e.g., CAD data, 3D data, digital blueprints, stereolithography data, or other data suitable for the various manufacturing technologies described herein). Different components of the system 100 can generate at least a portion of the manufacturing data used by the manufacturing system 124. The manufacturing data can include, without limitation, fabrication instructions (e.g., programs executable by additive manufacturing equipment, subtractive manufacturing equipment, etc.), 3D data, CAD data (e.g., CAD files), CAM data (e.g., CAM files), path data (e.g., print head paths, tool paths, etc.), material data, tolerance data, surface finish data (e.g., surface roughness data), regulatory data (e.g., FDA requirements, reimbursement data, etc.), or the like. The manufacturing system 124 can analyze the manufacturability of the implant design based on the received manufacturing data. The implant design can be finalized by altering geometries, surfaces, etc. and then generating manufacturing instructions. In some embodiments, the server 106 generates at least a portion of the manufacturing data, which is transmitted to the manufacturing system 124.

The manufacturing system 124 can generate CAM data, print data (e.g., powder bed print data, thermoplastic print data, photo resin data, etc.), or the like and can include additive manufacturing equipment, subtractive manufacturing equipment, thermal processing equipment, or the like. The additive manufacturing equipment can be 3D printers, stereolithography devices, digital light processing devices, fused deposition modeling devices, selective laser sintering devices, selective laser melting devices, electronic beam melting devices, laminated object manufacturing devices, powder bed printers, thermoplastic printers, direct material deposition devices, or inkjet photo resin printers, or like technologies. The subtractive manufacturing equipment can be CNC machines, electrical discharge machines, grinders, laser cutters, water jet machines, manual machines (e.g., milling machines, lathes, etc.), or like technologies. Both additive and subtractive techniques can be used to produce implants with complex geometries, surface finishes, material properties, etc. The generated fabrication instructions can be configured to cause the manufacturing system 124 to manufacture the patient-specific orthopedic implant that matches or is therapeutically the same as the patient-specific design. In some embodiments, the patient-specific medical device can include features, materials, and designs shared across designs to simplify manufacturing. For example, deployable patient-specific medical devices for different patients can have similar internal deployment mechanisms but have different deployed configurations. In some embodiments, the components of the patient-specific medical devices are selected from a set of available pre-fabricated components and the selected pre-fabricated components can be modified based on the fabrication instructions or data.

The manufacturing system 124, implant analyzer 131, and/or regulatory and reimbursement manager 119 can communicate directly with one another or via the communication network 104. The system 100 can perform one or more validation steps for a manufactured implant. The analyzer 131 can include one or more scanners, cameras, or imaging devices and can be incorporated into the manufacturing system 124 or other components of the system 100 and can scan the manufactured implant to, for example, identify manufacturing defects, confirm the implant meets one or regulatory requirements, etc. By analyzing implant characteristics (e.g., composition of the material, surface topology, etc.) and manufacturing parameters (e.g., composition of the material, temperature, speed of printing, manufacturing conditions, accuracy of printer, etc.), the system 100 can determine whether the implant should be implanted in a patient. If the implant is not acceptable, system 100 can determine manufacturing adjustments for the implant to be remanufactured. The analyzer 131 can be onsite manufacturing scanners positioned to scan implants during and/or after fabrication. In some embodiments, the analyzers 131 are offsite of the manufacturing location. For example, the analyzers 131 can be located at a healthcare provider (e.g., at a hospital, clinic, surgical suite, etc.) to allow quality control checking immediately prior to implantation, verification of regulatory compliance, etc.

The manufacturing system 124 can manufacture all or some of the components of a kit. The kit components can be selected based on requirement(s), including regulatory requirements, reimbursement requirements, or other requirements. Surgical kits can include one or more implants, instruments, instructions for use, and reusable and disposable components. The kit requirements can be retrieved from a database 151. The system 100 can synchronize the surgical plan with the requirements to generate patient-specific surgical kits meeting the requirements.

The treatment plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments the treatment planning module 118 generates control instructions configured to cause a surgical robot (e.g., robotic surgery systems, navigation systems, etc.) to partially or fully perform a surgical procedure. The control instructions can be transmitted to the robotic apparatus by the client computing device 102 and/or the server 106.

Following the treatment of the patient in accordance with the treatment plan, treatment progress can be monitored over one or more time periods to update the data analysis module 116 and/or treatment planning module 118. Post-treatment data can be added to the reference data stored in the database 110. The post-treatment data can be used to train machine learning models for developing patient-specific treatment plans, patient-specific medical devices, or combinations thereof.

It shall be appreciated that the components of the system 100 can be configured in many different ways. For example, in alternative embodiments, the database 110, the data analysis module 116 and/or the treatment planning module 118 can be components of the client computing device 102, rather than the server 106. As another example, the database 110 the data analysis module 116, and/or the treatment planning module 118 can be located across a plurality of different servers, computing systems, or other types of cloud-computing resources, rather than at a single server 106 or client computing device 102.

The system 100 can analyze patient data to identify types of treatments that can be performed and corresponding payment information (e.g., coverage by insurance, reimbursement coding, etc.). The system 100 can then develop candidate treatment plans based on the payment information, thereby tailoring treatments to maximize financial coverage. The regulatory and reimbursement manager 119 can communicate with the database 151 and can analyze regulatory information, insurance policies, reimbursement information, historical payment information (e.g., successful payment submissions, unsuccessful payment submissions, etc.), governmental regulatory information, etc. The regulatory information can include, without limitation, design constraints for implants (e.g., size, implant footprint, configuration, maximum size, minimize size), material information, etc. The reimbursement information can include, without limitation, medical reimbursement codes, insurance codes, healthcare provider codes, etc. The database 151 can store charge analysis based on region (e.g., pacific, mountain, central, Atlantic, etc.), urban/rural status, number of beds, standard total implant/device charge, standard total charge, new technology add-on payment (NTAP) overall amount, total agency (Medicare or third-party payor) payment. The charge analysis data can be considered when developing plans disclosed herein.

The database 151 can also store condition identifiers (e.g., CPT codes, ICD10 codes, X-codes, CPT codes, HCPCS codes, etc.), national coverage determinations (NCDs), local coverage determinations (LCDs), insurance policy information (e.g., local or national policies of private or public payors, such as Centers of Medicare and Medicaid Services (CMS), VA, etc.), reimbursements/pricing, schedules (e.g., Medicare Physician Fee Schedule (MPFS), Hospital Outpatient Prospective Payment System (HOPPS) schedule, ambulatory payment classification (APC), etc.), and other requirements for reimbursements. The kits can include one or more implants, instruments, instructions for use (IFU), and reusable and disposable components. In some embodiments, the system 100 can identify a surgical procedure and then retrieve requirements for reimbursement from the database 151. The system 100 can then synchronize the surgical plan with the reimbursement requirements to generate patient-specific surgical kits meeting the reimbursement requirements.

The treatment planning module 118 can communicate with the regulatory and reimbursement manager 119 to obtain reimbursement information. The display 122 can display a reimbursement data 123 and regulatory data 127 while the user reviews proposed pathology 129, a treatment plan 157, and implant(s) 161. The treatment plan 257 can be an interactive plan having a user input element 165 (e.g., one or more buttons, a dropdown menu, toggle, etc.) for modification and/or approval. The reimbursement data 123 and regulatory data 127 can be dynamically updated based on the user input. This allows a user to identify correlations between treatment and non-treatment considerations. Additionally, reports can be generated to facilitate with billing.

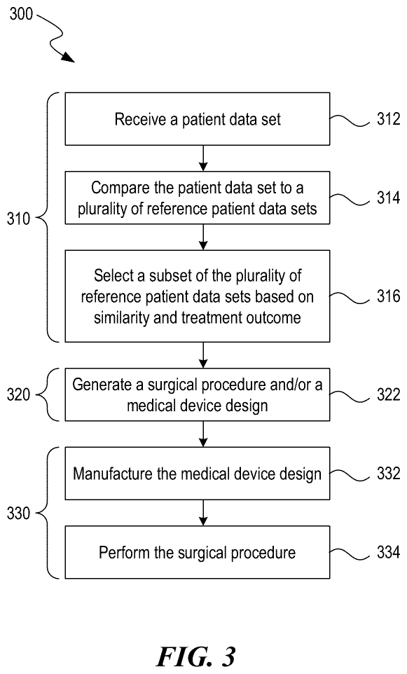

The system 100 is configured to design the physical patient-specific implants 154 for achieving the approved planned pathology 129. The regulatory and reimbursement manager 119 can also retrieve information regarding the patient's anatomy, such as pre-operative measurements, two- or three-dimensional images or models of the anatomy, and/or information regarding the biology, geometry, and/or mechanical properties of the anatomy. Example implant design is discussed in connection with FIGS. 3-13.

The system 100 of FIG. 1 can receive reimbursement information from the healthcare provider via, for example, a portal, a database, or other communication means. The system can then analyze the treatment plan and patient data to verify that the reimbursement information is acceptable. If the system identifies unacceptable reimbursement information (e.g., codes that do not match the patient data), the system can send an alert to the healthcare provider. In this manner, the system can analyze and check coding prior to submission by the healthcare provider. Additionally, if the system identifies inconsistencies or potentially incorrect reimbursement information, the system can generate recommended alternative reimbursement information with associated justification. In some embodiments, the system can generate and send a reimbursement report with a confidence level above a threshold level to the healthcare provider. This can help ensure that proper reimbursement information and correct coding is utilized.

Additionally, in some embodiments, the system 100 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like.

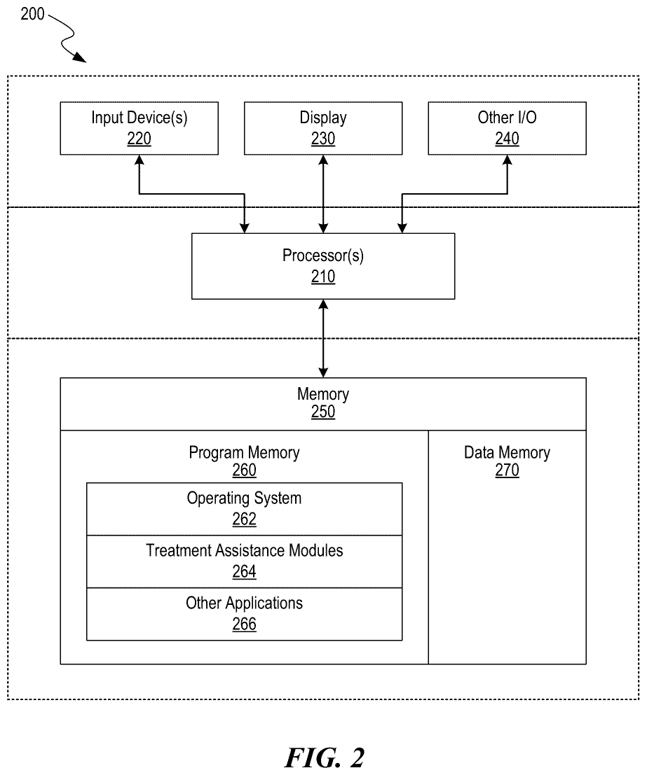

FIG. 2 illustrates a computing device 200 suitable for use in connection with the system 100 of FIG. 1, according to an embodiment. The computing device 200 can be incorporated in various components of the system 100 of FIG. 1, such as the client computing device 102 or the server 106. The computing device 200 includes one or more processors 210 (e.g., CPU(s), GPU(s), HPU(s), etc.). The processor(s) 210 can be a single processing unit or multiple processing units in a device or distributed across multiple devices. The processor(s) 210 can be coupled to other hardware devices, for example, with the use of a bus, such as a PCI bus or SCSI bus. The processor(s) 210 can be configured to execute one more computer-readable program instructions, such as program instructions to carry out of any of the methods described herein.

The computing device 200 can include one or more input devices 220 that provide input to the processor(s) 210, e.g., to notify it of actions from a user of the device 200. The actions can be mediated by a hardware controller that interprets the signals received from the input device and communicates the information to the processor(s) 210 using a communication protocol. Input device(s) 220 can include, for example, a mouse, a keyboard, a touchscreen, an infrared sensor, a touchpad, a wearable input device, a camera- or image-based input device, a microphone, or other user input devices.

The computing device 200 can include a display 230 used to display various types of output, such as text, models, virtual procedures, surgical plans, implants, graphics, and/or images (e.g., images with voxels indicating radiodensity units or Hounsfield units representing the density of the tissue at a location). In some embodiments, the display 230 provides graphical and textual visual feedback to a user. The processor(s) 210 can communicate with the display 230 via a hardware controller for devices. In some embodiments, the display 230 includes the input device(s) 220 as part of the display 230, such as when the input device(s) 220 include a touchscreen or is equipped with an eye direction monitoring system. In alternative embodiments, the display 230 is separate from the input device(s) 220. Examples of display devices include an LCD display screen, an LED display screen, a projected, holographic, or augmented reality display (e.g., a heads-up display device or a head-mounted device), and so on.

Optionally, other I/O devices 240 can also be coupled to the processor(s) 210, such as a network card, video card, audio card, USB, firewire or other external device, camera, printer, speakers, CD-ROM drive, DVD drive, disk drive, or Blu-Ray device. Other I/O devices 240 can also include input ports for information from directly connected medical equipment such as imaging apparatuses, including MRI machines, X-Ray machines, CT machines, etc. Other I/O devices 240 can further include input ports for receiving data from these types of machine from other sources, such as across a network or from previously captured data, for example, stored in a database.

In some embodiments, the computing device 200 also includes a communication device (not shown) capable of communicating wirelessly or wire-based with a network node. The communication device can communicate with another device or a server through a network using, for example, TCP/IP protocols. The computing device 200 can utilize the communication device to distribute operations across multiple network devices, including imaging equipment, manufacturing equipment, etc.

The computing device 200 can include memory 250, which can be in a single device or distributed across multiple devices. Memory 250 includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. In some embodiments, the memory 250 is a non-transitory computer-readable storage medium that stores, for example, programs, software, data, or the like. In some embodiments, memory 250 can include program memory 260 that stores programs and software, such as an operating system 262, one or more treatment assistance modules 264, and other application programs 266. The treatment assistance module(s) 264 can include one or more modules configured to perform the various methods described herein (e.g., the data analysis module 116 and/or treatment planning module 118 described with respect to FIG. 1). Memory 250 can also include data memory 270 that can include, e.g., reference data, configuration data, settings, user options or preferences, etc., which can be provided to the program memory 260 or any other element of the computing device 200.

FIG. 3 is a flow diagram illustrating a method 300 for providing patient-specific medical care, according to an embodiment. The method 300 can include a data phase 310, a modeling phase 320, and an execution phase 330. The data phase 310 can include collecting data of a patient to be treated (e.g., pathology data), and comparing the patient data to reference data (e.g., prior patient data such as pathology, surgical, and/or outcome data). For example, a patient data set can be received (block 312). The patient data set can be compared to a plurality of reference patient data sets (block 314), e.g., in order to identify one or more similar patient data sets in the plurality of reference patient data sets. Each of the plurality of reference patient data sets can include data representing one or more of age, gender, BMI, lumbar lordosis, Cobb angle(s), pelvic incidence, disc height, coronal offset distance, segment flexibility, LL-PI is greater than predetermined degrees (e.g., 5 degrees, 10 degrees, etc.), LL-PI mismatch (e.g., age-adjusted), sagittal vertical axis offset distance, coronal offset distance, coronal angle, bone quality, rotational displacement, or treatment level of the spine.

A subset of the plurality of reference patient data sets can be selected (block 316), e.g., based on similarity to the patient data set and/or treatment outcomes of the corresponding reference patients. For example, a similarity score can be generated for each reference patient data set, based on the comparison of the patient data set and the reference patient data set. The similarity score can represent a statistical correlation between the patient data and the reference patient data set. One or more similar patient data sets can be identified based, at least partly, on the similarity score.

In some embodiments, each patient data set of the selected subset includes and/or is associated with data indicative of a favorable treatment outcome (e.g., a favorable treatment outcome based on a single target outcome, aggregate outcome score, outcome thresholding). The data can include, for example, data representing one or more of corrected anatomical metrics, presence of fusion, health related quality of life, activity level, or complications. In some embodiments, the data is or includes an outcome score, which can be calculated based on a single target outcome, an aggregate outcome, and/or an outcome threshold.

Optionally, the data analysis phase 310 can include identifying or determining, for at least one patient data set of the selected subset (e.g., for at least one similar patient data set), surgical procedure data and/or medical device design data associated with the favorable treatment outcome. The surgical procedure data can include data representing one or more of a surgical approach, a corrective maneuver, a bony resection, or implant placement. The at least one medical device design can include data representing one or more of physical properties, mechanical properties, or biological properties of a corresponding medical device. In some embodiments, the at least one patient-specific medical device design includes a design for an implant or an implant delivery instrument.

In the modeling phase 320, a surgical procedure and/or medical device design is generated (block 322). The generating step can include developing at least one predictive model based on the patient data set and/or selected subset of reference patient data sets (e.g., using statistics, machine learning, neural networks, AI, or the like). The predictive model can be configured to generate the surgical procedure and/or medical device design.

In some embodiments, the predictive model includes one or more trained machine learning models that generate, at least partly, the surgical procedure and/or medical device design. For example, the trained machine learning model(s) can determine a plurality of candidate surgical procedures and/or medical device designs for treating the patient. Each surgical procedure can be associated with a corresponding medical device design. In some embodiments, the surgical procedures and/or medical device designs are determined based on surgical procedure data and/or medical device design data associated with favorable outcomes, as previously described with respect to the data analysis phase 310. For each surgical procedure and/or corresponding medical device design, the trained machine learning model(s) can calculate a probability of achieving a target outcome (e.g., favorable or desired outcome) for the patient. The trained machine learning model(s) can then select at least one surgical procedure and/or corresponding medical device design based, at least partly, on the calculated probabilities.

The execution phase 330 can include manufacturing the medical device design (block 332). In some embodiments, the medical device design is manufactured by a manufacturing system configured to perform one or more of additive manufacturing, 3D printing, stereolithography, digital light processing, fused deposition modeling, selective laser sintering, selective laser melting, electronic beam melting, laminated object manufacturing, powder bed printing, thermoplastic printing, direct material deposition, or inkjet photo resin printing. The execution phase 330 can optionally include generating fabrication instructions configured to cause the manufacturing system to manufacture a medical device having the medical device design.

The execution phase 330 can include performing the surgical procedure (block 334). The surgical procedure can involve implanting a medical device having the medical device design into the patient. The surgical procedure can be performed manually, by a surgical robot, or a combination thereof. In embodiments where the surgical procedure is performed by a surgical robot, the execution phase 330 can include generating control instructions configured to cause the surgical robot to perform, at least partly, the patient-specific surgical procedure.

The method 300 can be implemented and performed in various ways. In some embodiments, one or more steps of the method 300 (e.g., the data phase 310 and/or the modeling phase 320) can be implemented as computer-readable instructions stored in memory and executable by one or more processors of any of the computing devices and systems described herein (e.g., the system 100), or a component thereof (e.g., the client computing device 102 and/or the server 106). Alternatively, one or more steps of the method 300 (e.g., the execution phase 330) can be performed by a healthcare provider (e.g., physician, surgeon), a robotic apparatus (e.g., a surgical robot), a manufacturing system (e.g., manufacturing system 124), or a combination thereof. In some embodiments, one or more steps of the method 300 are omitted (e.g., the execution phase 330).

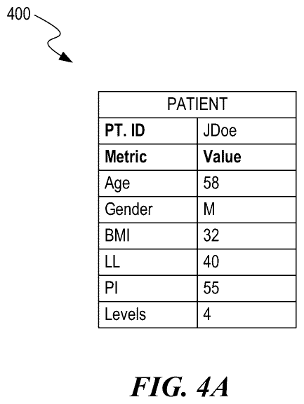

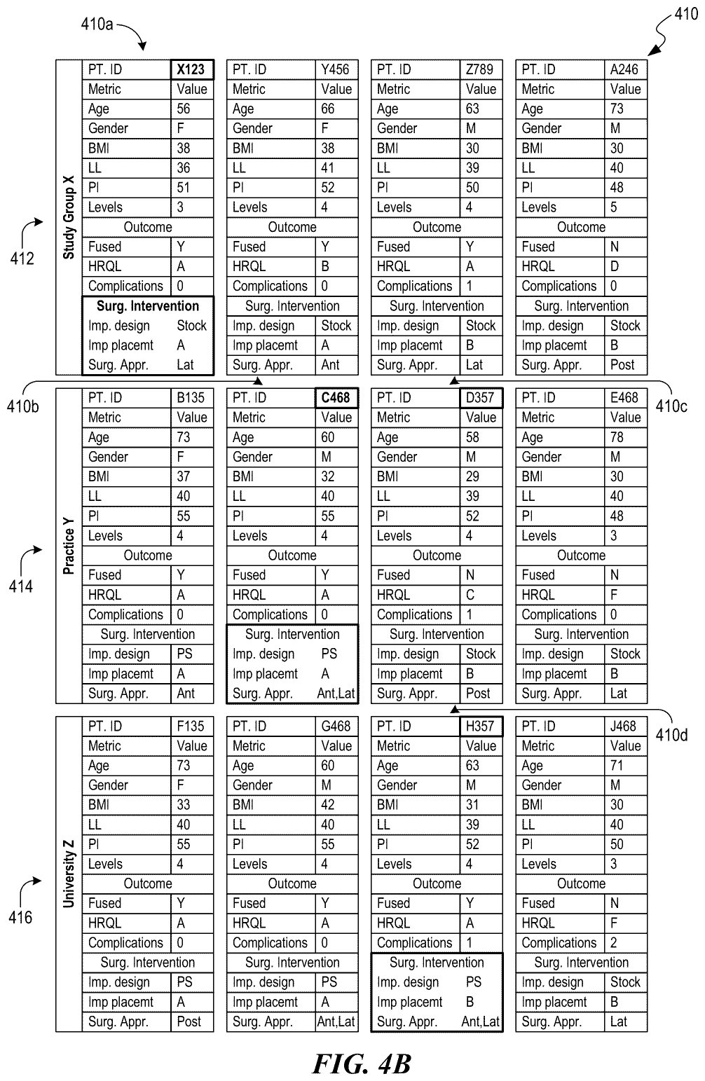

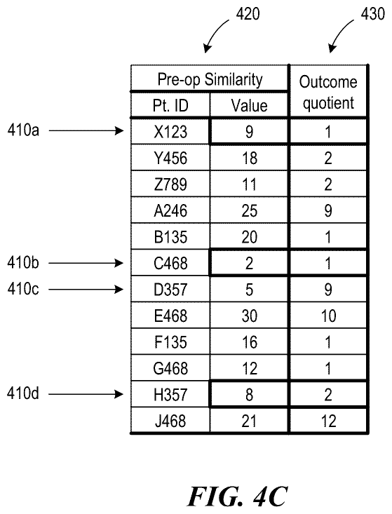

FIGS. 4A-4C illustrate exemplary data sets that may be used and/or generated in connection with the methods described herein (e.g., the data analysis phase 310 described with respect to FIG. 3), according to an embodiment. FIG. 4A illustrates a patient data set 400 of a patient to be treated. The patient data set 400 can include a patient ID and a plurality of pre-operative patient metrics (e.g., age, gender, BMI, lumbar lordosis (LL), pelvic incidence (PI), and treatment levels of the spine (levels)). FIG. 4B illustrates a plurality of reference patient data sets 410. In the depicted embodiment, the reference patient data sets 410 include a first subset 412 from a study group (Study Group X), a second subset 414 from a practice database (Practice Y), and a third subset 416 from an academic group (University Z). In alternative embodiments, the reference patient data sets 410 can include data from other sources, as previously described herein. Each reference patient data set can include a patient ID, a plurality of pre-operative patient metrics (e.g., age, gender, BMI, lumbar lordosis (LL), pelvic incidence (PI), and treatment levels of the spine (levels)), treatment outcome data (Outcome) (e.g., presence of fusion (fused), HRQL, complications), and treatment procedure data (Surg. Intervention) (e.g., implant design, implant placement, surgical approach).

FIG. 4C illustrates comparison of the patient data set 400 to the reference patient data sets 410. As previously described, the patient data set 400 can be compared to the reference patient data sets 410 to identify one or more similar patient data sets from the reference patient data sets. In some embodiments, the patient metrics from the reference patient data sets 410 are converted to numeric values and compared the patient metrics from the patient data set 400 to calculate a similarity score 420 (“Pre-op Similarity”) for each reference patient data set. Reference patient data sets having a similarity score below a threshold value can be considered to be similar to the patient data set 400. For example, in the depicted embodiment, reference patient data set 410a has a similarity score of 9, reference patient data set 410b has a similarity score of 2, reference patient data set 410c has a similarity score of 5, and reference patient data set 410d has a similarity score of 8. Because each of these scores are below the threshold value of 10, reference patient data sets 410a-d are identified as being similar patient data sets.

The treatment outcome data of the similar patient data sets 410a-d can be analyzed to determine surgical procedures and/or implant designs with the highest probabilities of success. For example, the treatment outcome data for each reference patient data set can be converted to a numerical outcome score 430 (“Outcome Quotient”) representing the likelihood of a favorable outcome. In the depicted embodiment, reference patient data set 410a has an outcome score of 1, reference patient data set 410b has an outcome score of 1, reference patient data set 410c has an outcome score of 9, and reference patient data set 410d has an outcome score of 2. In embodiments where a lower outcome score correlates to a higher likelihood of a favorable outcome, reference patient data sets 410a, 410b, and 410d can be selected. The treatment procedure data from the selected reference patient data sets 410a, 410b, and 410d can then be used to determine at least one surgical procedure (e.g., implant placement, surgical approach) and/or implant design that is likely to produce a favorable outcome for the patient to be treated.

In some embodiments, a method for providing medical care to a patient is provided. The method can include comparing a patient data set to reference data. The patient data set and reference data can include any of the data types described herein. The method can include identifying and/or selecting relevant reference data (e.g., data relevant to treatment of the patient, such as data of similar patients and/or data of similar treatment procedures), using any of the techniques described herein. A treatment plan can be generated based on the selected data, using any of the techniques described herein. The treatment plan can include one or more treatment procedures (e.g., surgical procedures, instructions for procedures, models or other virtual representations of procedures), one or more medical devices (e.g., implanted devices, instruments for delivering devices, surgical kits), or a combination thereof.

In some embodiments, a system for generating a medical treatment plan is provided. The system can compare a patient data set to a plurality of reference patient data sets, using any of the techniques described herein. A subset of the plurality of reference patient data sets can be selected, e.g., based on similarity and/or treatment outcome, or any other technique as described herein. A medical treatment plan can be generated based at least in part on the selected subset, using any of the techniques described herein. The medical treatment plan can include one or more treatment procedures, one or more medical devices, or any of the other aspects of a treatment plan described herein, or combinations thereof.

In further embodiments, a system is configured to use historical patient data. The system can select historical patient data to develop or select a treatment plan, design medical devices, or the like. Historical data can be selected based on one or more similarities between the present patient and prior patients to develop a prescriptive treatment plan designed for desired outcomes. The prescriptive treatment plan can be tailored for the present patient to increase the likelihood of the desired outcome. In some embodiments, the system can analyze and/or select a subset of historical data to generate one or more treatment procedures, one or more medical devices, or a combination thereof. In some embodiments, the system can use subsets of data from one or more groups of prior patients, with favorable outcomes, to produce a reference historical data set used to, for example, design, develop or select the treatment plan, medical devices, or combinations thereof.

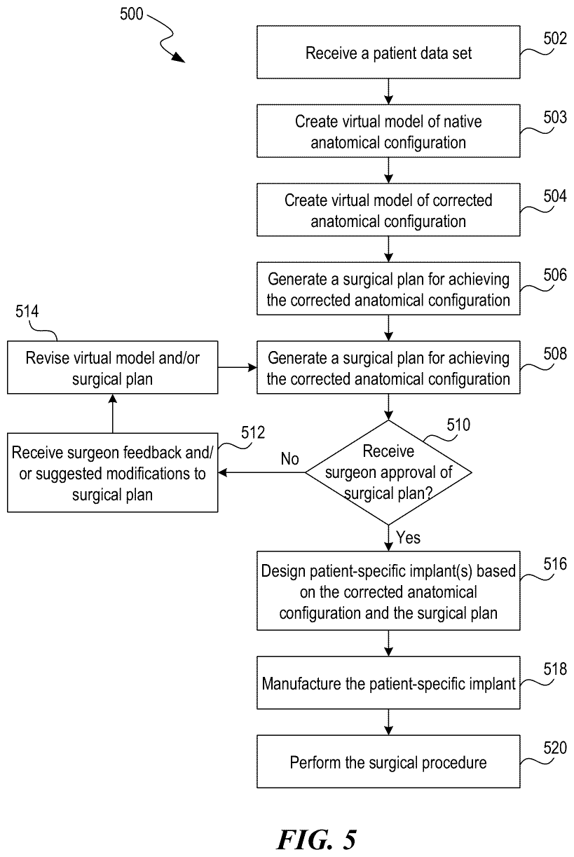

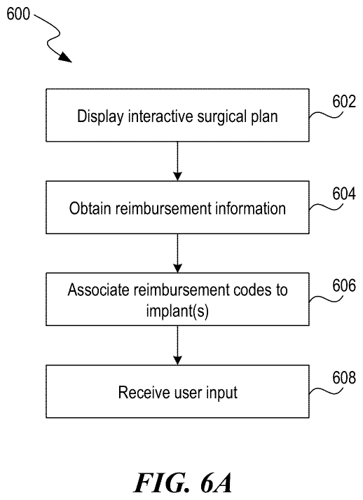

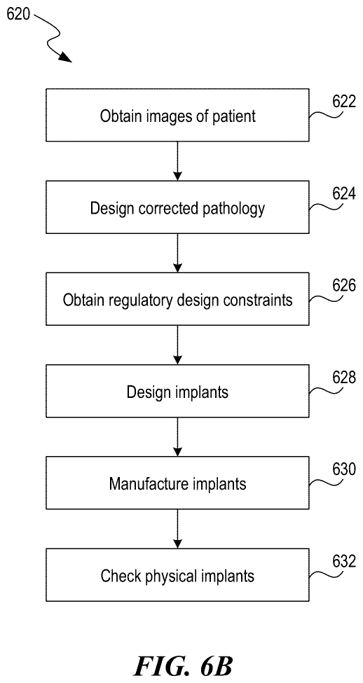

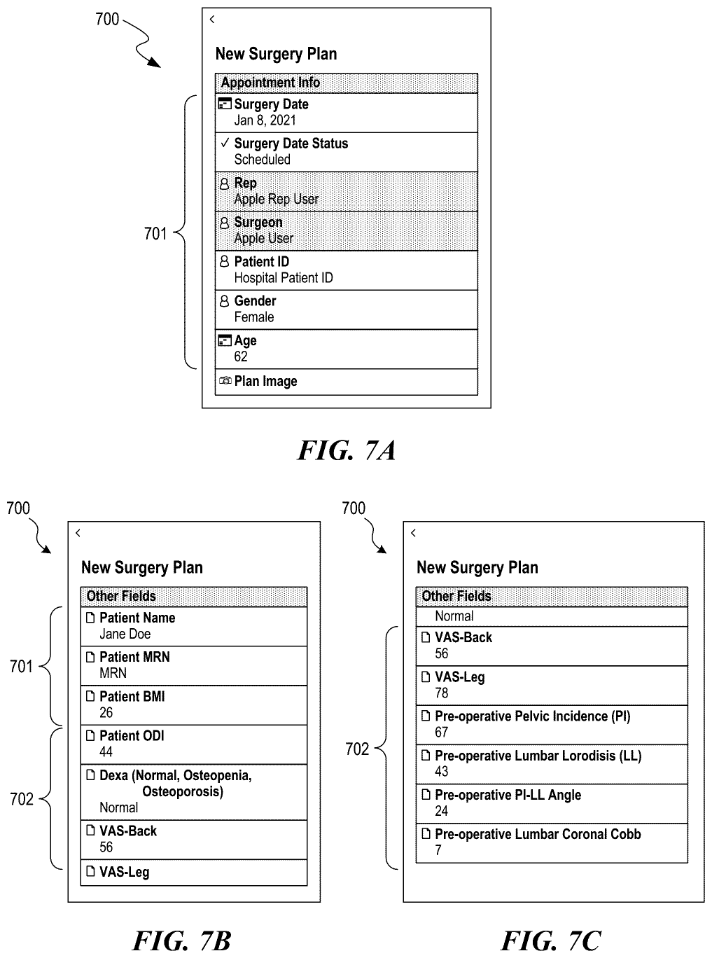

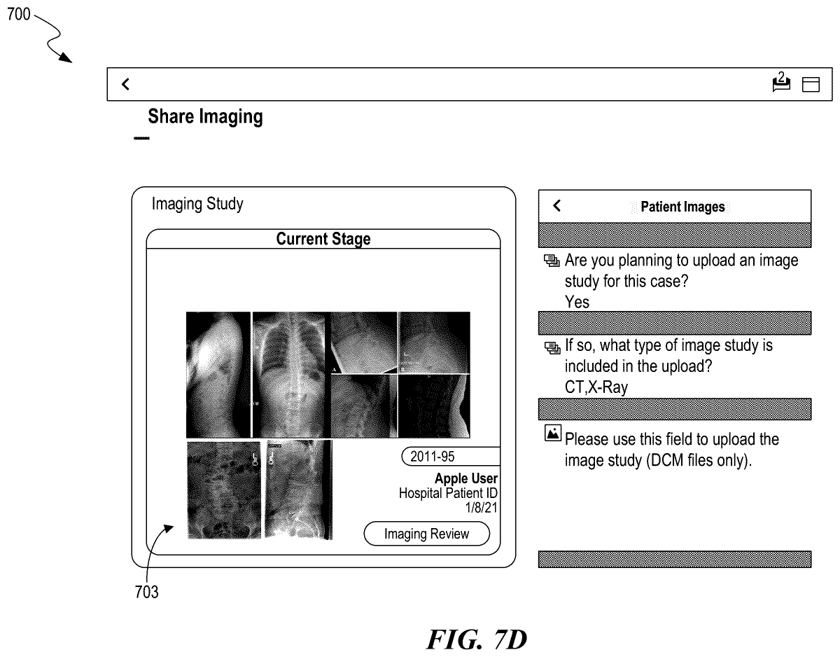

FIG. 5 is a flow diagram illustrating a method 500 for providing patient-specific medical care, according to another embodiment of the present technology. The method 500 can begin in step 502 by receiving a patient data set for a particular patient in need of medical treatment. The patient data set can include data representative of the patient's condition, anatomy, pathology, symptoms, medical history, preferences, and/or any other information or parameters relevant to the patient. For example, the patient data set 808 can include surgical intervention data, treatment outcome data, progress data (e.g., surgeon notes), patient feedback (e.g., feedback acquired using quality of life questionnaires, surveys), clinical data, patient information (e.g., demographics, sex, age, height, weight, type of pathology, occupation, activity level, tissue information, health rating, comorbidities, health related quality of life (HRQL)), vital signs, diagnostic results, medication information, allergies, diagnostic equipment information (e.g., manufacturer, model number, specifications, user-selected settings/configurations, etc.) or the like. The patient data set can also include image data, such as camera images, Magnetic Resonance Imaging (MRI) images, ultrasound images, Computerized Aided Tomography (CAT) scan images, Positron Emission Tomography (PET) images, X-Ray images, and the like. In some embodiments, the patient data set includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine. The patient data set can be received at a server, computing device, or other computing system. For example, in some embodiments the patient data set can be received by the server 106 shown in FIG. 1. In some embodiments, the computing system that receives the patient data set in step 502 also stores one or more software modules (e.g., the data analysis module 116 and/or the treatment planning module 118, shown in FIG. 1, or additional software modules for performing various operations of the method 500). Additional details for collecting and receiving the patient data set are described below with respect to FIGS. 6-7D.

In some embodiments, the received patient data set can include disease metrics such as lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.) and/or pelvic parameters. The disease metrics can include micro-measurements (e.g., metrics associated with specific or individual segments of the patient's spine) and/or macro-measurements (e.g., metrics associated with multiple segments of the patient's spine). In some embodiments, the disease metrics are not included in the patient data set, and the method 500 includes determining (e.g., automatically determining) one or more of the disease metrics based on the patient image data, as described below.

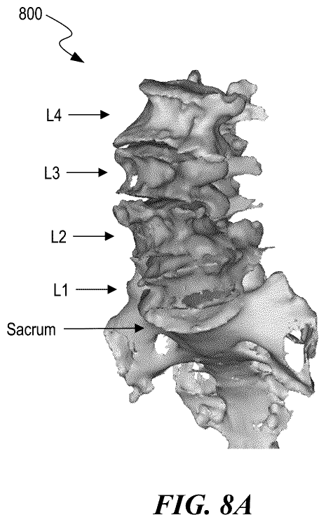

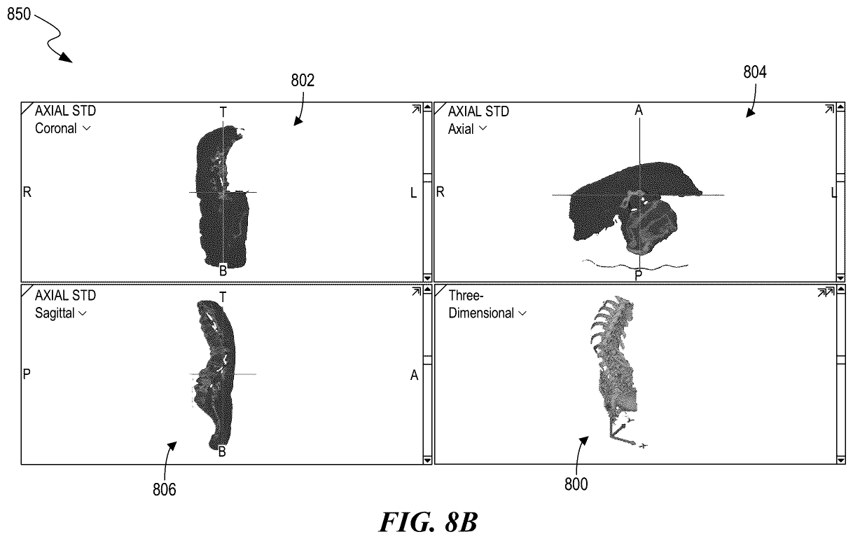

Once the patient data set is received in step 502, the method 500 can continue in step 503 by creating a virtual model of the patient's native anatomical configuration (also referred to as “pre-operative anatomical configuration”). The virtual model can be based on the image data included in the patient data set received in step 502. For example, the same computing system that received the patient data set in step 502 can analyze the image data in the patient data set to generate a virtual model of the patient's native anatomical configuration. The virtual model can be a two- or three-dimensional visual representation of the patient's native anatomy. The virtual model can include one or more regions of interest, and may include some or all of the patient's anatomy within the regions of interest (e.g., any combination of tissue types including, but not limited to, bony structures, cartilage, soft tissue, vascular tissue, nervous tissue, etc.). As a non-limiting example, the virtual model can include a visual representation of the patient's spinal cord region, including some or all of the sacrum, lumbar region, thoracic region, and/or cervical region. In some embodiments, the virtual model includes soft tissue, cartilage, and other non-bony structures. In other embodiments, the virtual model only includes the patient's bony structures. An example of a virtual model of the native anatomical configuration is described below with respect to FIGS. 8A and 8B. In some embodiments, the method 500 can optionally omit creating a virtual model of the patient's native anatomy in step 503, and proceed directly from step 502 to step 504.

In some embodiments, the computing system that generated the virtual model in step 502 can also determine (e.g., automatically determine or measure) one or more disease metrics of the patient based on the virtual model. For example, the computing system may analyze the virtual model to determine the patient's pre-operative lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.) and/or pelvic parameters. The disease metrics can include micro-measurements (e.g., metrics associated with specific or individual segments of the patient's spine) and/or macro-measurements (e.g., metrics associated with multiple segments of the patient's spine).

The method 500 can continue in step 504 by creating a virtual model of a corrected anatomical configuration (which can also be referred to herein as the “planned configuration,” “optimized geometry,” “post-operative anatomical configuration,” or “target outcome”) for the patient. For example, the computing system can, using the analysis procedures described previously, determine a “corrected” or “optimized” anatomical configuration for the particular patient that represents an ideal surgical outcome for the particular patient. This can be done, for example, by analyzing a plurality of reference patient data sets to identify post-operative anatomical configurations for similar patients who had a favorable post-operative outcome, as previously described in detail with respect to FIGS. 1-4C (e.g., based on similarity of the reference patient data set to the patient data set and/or whether the reference patient had a favorable treatment outcome). This may also include applying one or more mathematical rules defining optimal anatomical outcomes (e.g., positional relationships between anatomic elements) and/or target (e.g., acceptable) post-operative metrics/design criteria (e.g., adjust anatomy so that the post-operative sagittal vertical axis is less than 7 mm, the post-operative Cobb angle less than 10 degrees, etc.). Target post-operative metrics can include, but are not limited to, target coronal parameters, target sagittal parameters, target pelvic incidence angle, target Cobb angle, target shoulder tilt, target iliolumbar angle, target coronal balance, target Cobb angle, target lordosis angle, and/or a target intervertebral space height. The different between the native anatomical configuration and the corrected anatomical configuration may be referred to as a “patient-specific correction” or “target correction.”

Once the corrected anatomical configuration is determined, the computing system can generate a two- or three-dimensional visual representation of the patient's anatomy with the corrected anatomical configuration. As with the virtual model created in step 503, the virtual model of the patient's corrected anatomical configuration can include one or more regions of interest, and may include some or all of the patient's anatomy within the regions of interest (e.g., any combination of tissue types including, but not limited to, bony structures, cartilage, soft tissue, vascular tissue, nervous tissue, etc.). As a non-limiting example, the virtual model can include a visual representation of the patient's spinal cord region in a corrected anatomical configuration, including some or all of the sacrum, lumbar region, thoracic region, and/or cervical region. In some embodiments, the virtual model includes soft tissue, cartilage, and other non-bony structures. In other embodiments, the virtual model only includes the patient's bony structures. An example of a virtual model of the native anatomical configuration is described below with respect to FIGS. 9A-1-9B-2.

In step 504, images of the patient can be segmented to isolate separate anatomic elements of the anatomy of interest. The spatial relationships between the isolated anatomic elements can be modified to generate a target or corrected patient pathology. The modifications can be selected based on regulatory criteria, financial parameters, etc. Other techniques can be used to generate anatomical configurations based on the available patient data.

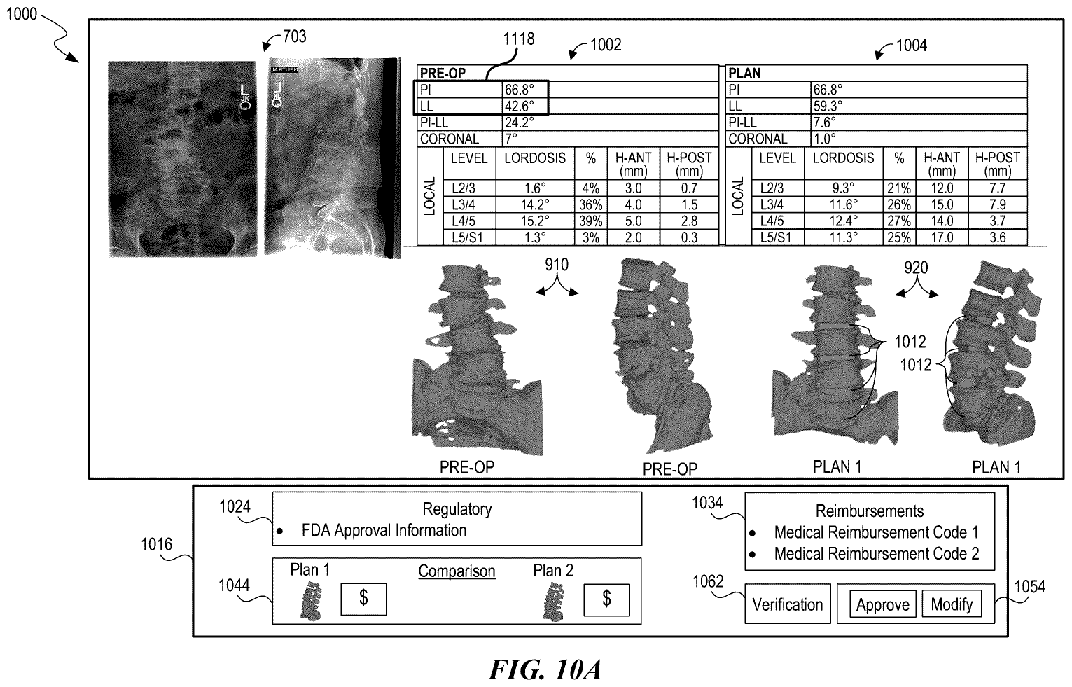

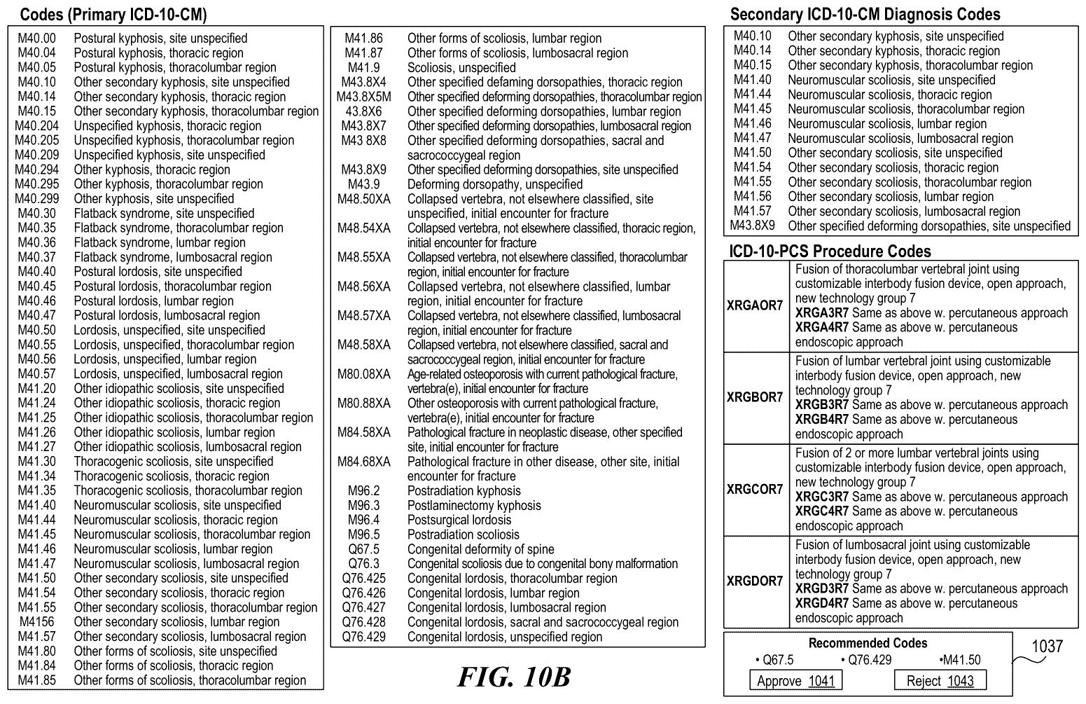

The method 500 can continue in step 506 by generating (e.g., automatically generating) a surgical plan for achieving the corrected anatomical configuration shown by the virtual model. The surgical plan can include pre-operative plans, operative plans, post-operative plans, and/or specific spine metrics associated with the optimal surgical outcome. For example, the surgical plans can include a specific surgical procedure for achieving the corrected anatomical configuration. In the context of spinal surgery, the surgical plan may include a specific fusion surgery (e.g., PLIF, ALIF, TLIF, LLIF, DLIF, XLIF, etc.) across a specific range of vertebral levels (e.g., L1-L4, L1-5, L3-T12, etc.). Of course, other surgical procedures may be identified for achieving the corrected anatomical configuration, such as non-fusion surgical approaches and orthopedic procedures for other areas of the patient. The surgical plan may also include one or more expected spine metrics (e.g., lumbar lordosis, Cobb angles, coronal parameters, sagittal parameters, and/or pelvic parameters) corresponding to the expected post-operative patient anatomy. The surgical plan can be generated by the same or different computing system that created the virtual model of the corrected anatomical configuration. In some embodiments, the surgical plan can also be based on one or more reference patient data sets as previously described with respect to FIGS. 1-4C. In some embodiments, the surgical plan can also be based at least in part on surgeon-specific preferences and/or outcomes associated with a specific surgeon performing the surgery. In some embodiments, more than one surgical plan is generated in step 506 to provide a surgeon with multiple options. An example of a surgical plan is described below with respect to FIG. 10.

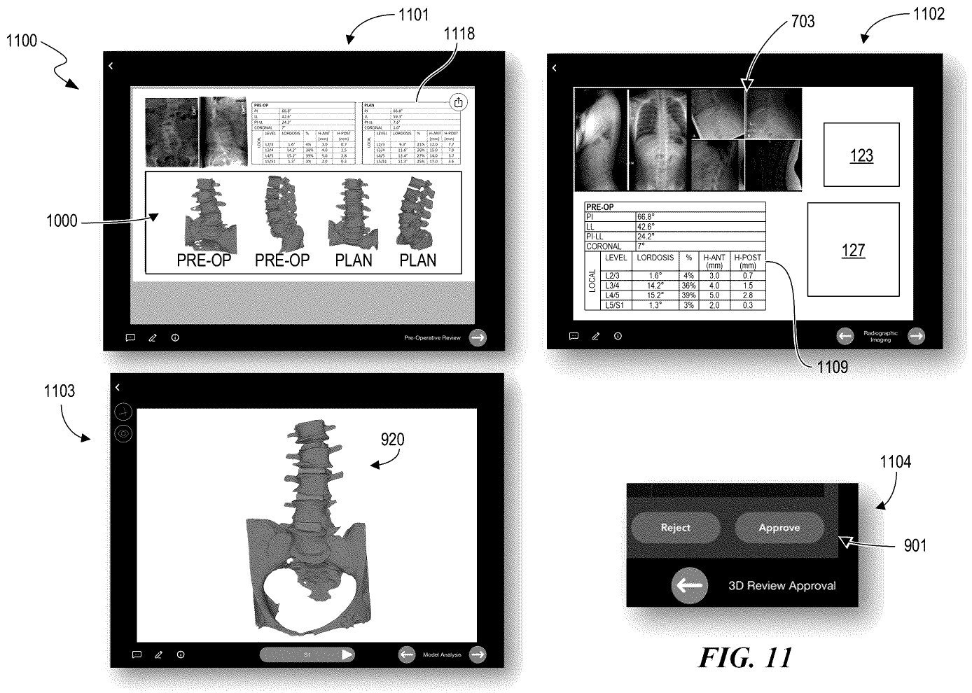

After the virtual model of the corrected anatomical configuration is created in step 504 and the surgical plan is generated in step 506, the method 500 can continue in step 508 by transmitting the virtual model of the corrected anatomical configuration and the surgical plan, including interactive surgical plans, for surgeon review. In some embodiments, the virtual model and the surgical plan are transmitted as a surgical plan report, an example of which is described with respect to FIG. 11. In some embodiments, the same computing system used in steps 502-506 can transmit the virtual model and surgical plan to a computing device for surgeon review (e.g., the client computing device 102 described in FIG. 1). This can include directly transmitting the virtual model and the surgical plan to the computing device or uploading the virtual model and the surgical plan to a cloud or other storage system for subsequent downloading. Although step 508 describes transmitting the surgical plan and the virtual model to the surgeon, one skilled in the art will appreciate from the disclosure herein that images of the virtual model may be included in the surgical plan transmitted to the surgeon, and that the actual model need not be included (e.g., to decrease the file size being transmitted). Additionally, the information transmitted to the surgeon in step 508 may include the virtual model of the patient's native anatomical configuration (or images thereof) in addition to the virtual model of the corrected anatomical configuration. In embodiments in which more than one surgical plan is generated in step 506, the method 500 can include transmitting more than one surgical plan to the surgeon for review and selection.

The surgeon can review the virtual model and surgical plan and, in step 510, either approve or reject the surgical plan (or, if more than one surgical plan is provided in step 508, select one of the provided surgical plans). If the surgeon does not approve the surgical plan in step 510, the surgeon can optionally provide feedback and/or suggested modifications to the surgical plan (e.g., by adjusting the virtual model or changing one or more aspects about the plan). Accordingly, the method 500 can include receiving (e.g., via the computing system) the surgeon feedback and/or suggested modifications. If surgeon feedback and/or suggested modifications are received in step 512, the method 500 can continue in step 514 by revising (e.g., automatically revising via the computing system) the virtual model and/or surgical plan based at least in part on the surgeon feedback and/or suggested modifications received in step 512. In some embodiments, the surgeon does not provide feedback and/or suggested modifications if they reject the surgical plan. In such embodiments, step 512 can be omitted, and the method 500 can continue in step 514 by revising (e.g., automatically revising via the computing system) the virtual model and/or the surgical plan by selecting new and/or additional reference patient data sets. The revised virtual model and/or surgical plan can then be transmitted to the surgeon for review. Steps 508, 510, 512, and 514 can be repeated as many times as necessary until the surgeon approves the surgical plan. Although described as the surgeon reviewing, modifying, approving, and/or rejecting the surgical plan, in some embodiments the surgeon can also review, modify, approve, and/or reject the corrected anatomical configuration shown via the virtual model.

Once surgeon approval of the surgical plan is received in step 510, the method 500 can continue in step 516 by designing (e.g., via the same computing system that performed steps 502-514) a patient-specific implant based on the corrected anatomical configuration and the surgical plan. The implant(s) (e.g., implants 154 or 161 of FIG. 1) can be designed by mapping a negative space between the anatomic elements and filling at least a portion of the negative space with a medical reimbursable virtual implant according to insurance information, regulatory information, reimbursement information, combinations thereof, or the like. U.S. application Ser. No. 16/569,494 discloses techniques for generating corrected patient pathologies, mapping spaces, designing implants, and manufacturing implants. U.S. application Ser. No. 16/569,494 is incorporated by reference in its entirety.

The patient-specific implant can be specifically designed such that, when it is implanted in the particular patient, it directs the patient's anatomy to occupy the corrected anatomical configuration (e.g., transforming the patient's anatomy from the native anatomical configuration to the corrected anatomical configuration). The patient-specific implant can be designed such that, when implanted, it causes the patient's anatomy to occupy the corrected anatomical configuration for the expected service life of the implant (e.g., 5 years or more, 10 years or more, 20 years or more, 50 years or more, etc.). In some embodiments, the patient-specific implant is designed solely based on the virtual model of the corrected anatomical configuration and/or without reference to pre-operative patient images.

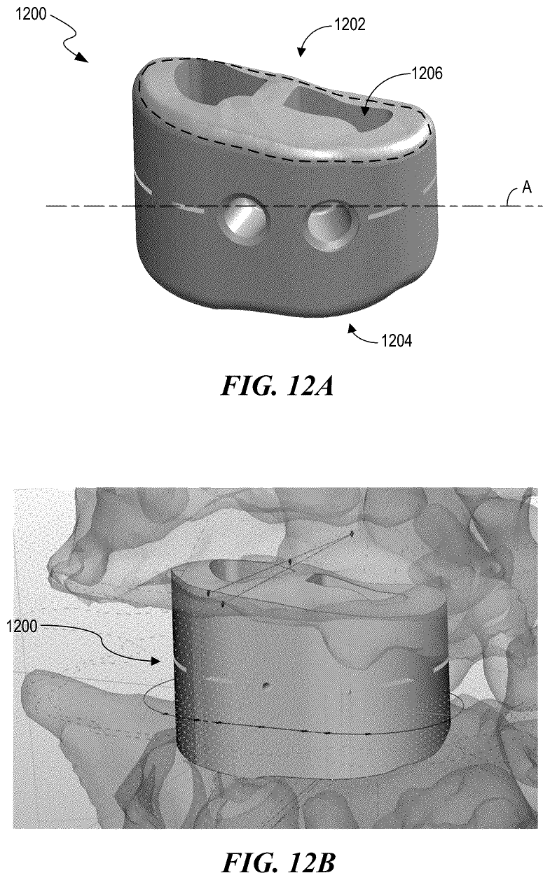

The patient-specific implant can be any of the implants described herein or in any patent references incorporated by reference herein. For example, the patient-specific implant can include one or more of screws (e.g., bone screws, spinal screws, pedicle screws, facet screws), interbody implant devices (e.g., intervertebral implants), cages, plates, rods, discs, fusion devices, spacers, rods, expandable devices, stents, brackets, ties, scaffolds, fixation device, anchors, nuts, bolts, rivets, connectors, tethers, fasteners, joint replacements (e.g., artificial discs), hip implants, or the like. A patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include implant shape, size, material, and/or effective stiffness (e.g., lattice density, number of struts, location of struts, etc.). An example of a patient-specific implant designed via the method 500 is described below with respect to FIGS. 12A and 12B.

In some embodiments, designing the implant in step 516 can optionally include generating fabrication instructions for manufacturing the implant. For example, the computing system may generate computer-executable fabrication instructions that that, when executed by a manufacturing system, cause the manufacturing system to manufacture the implant. For example, a virtual 3D model of the one or more patient-specific implants can be created based on filling of negative spaces between anatomical elements of the corrected patient pathology. The virtual 3D model can be converted into 3D fabrication data for manufacturing the one or more patient-specific implants.

In some embodiments, the patient-specific implant is designed in step 516 only after the surgeon has reviewed and approved the virtual model with the corrected anatomical configuration and the surgical plan. Accordingly, in some embodiments, the implant design is neither transmitted to the surgeon with the surgical plan in step 508, nor manufactured before receiving surgeon approval of the surgical plan. Without being bound by theory, waiting to design the patient-specific implant until after the surgeon approves the surgical plan may increase the efficiency of the method 500 and/or reduce the resources necessary to perform the method 500.

The method 500 can continue in step 518 by manufacturing the patient-specific implant. The implant can be manufactured using additive manufacturing techniques, such as 3D printing, stereolithography, digital light processing, fused deposition modeling, selective laser sintering, selective laser melting, electronic beam melting, laminated object manufacturing, powder bed printing, thermoplastic printing, direct material deposition, or inkjet photo resin printing, or like technologies, or combination thereof. Alternatively or additionally, the implant can be manufactured using subtractive manufacturing techniques, such as CNC machining, electrical discharge machining (EDM), grinding, laser cutting, water jet machining, manual machining (e.g., milling, lathe/turning), or like technologies, or combinations thereof. The implant may be manufactured by any suitable manufacturing system (e.g., the manufacturing system 124 shown in FIG. 1). In some embodiments, the implant is manufactured by the manufacturing system executing the computer-readable fabrication instructions generated by the computing system in step 516.

Once the implant is manufactured in step 518, the method 500 can continue in step 520 by implanting the patient-specific implant into the patient. The surgical procedure can be performed manually, by a robotic surgical platform (e.g., a surgical robot), or a combination thereof. In embodiments in which the surgical procedure is performed at least in part by a robotic surgical platform, the surgical plan can include computer-readable control instructions configured to cause the surgical robot to perform, at least partly, the patient-specific surgical procedure.

The method 500 can be implemented and performed in various ways. In some embodiments, steps 502-516 can be performed by a computing system associated with a first entity, step 518 can be performed by a manufacturing system associated with a second entity, and step 520 can be performed by a surgical provider, surgeon, and/or robotic surgical platform associated with a third entity. Any of the foregoing steps may also be implemented as computer-readable instructions stored in memory and executable by one or more processors of the associated computing system(s).

FIG. 6A is a flow diagram illustrating a method 600 for managing reimbursements, according to another embodiment of the present technology. The method 600 can begin in step 602 by displaying an interactive plan generated based on patient data. A patient-specific interactive surgical plan (e.g., plan 132 of FIG. 1 or plan 1000 of FIG. 10A-10B) includes a viewable planned pathology for the patient and is configured to receive user input. The pre-operative pathology can be used to validate a diagnosis, qualifying conditions for treatment, or the like based on pre-operative measurements, such as lumbar lordosis, Cobb angle(s), pelvic incidence, disc height(s), coronal offset distance, segment flexibility, LL-PI is greater than predetermined degrees (e.g., 5 degrees, 10 degrees, 15 degrees, etc.), LL-PI mismatch (e.g., age-adjusted), sagittal vertical axis offset distance, coronal offset distance, coronal angle, bone quality, and other metrics disclosed herein. Example displayed interactive plans and viewable pathologies are discussed in connection with FIGS. 7A-11.

The method 600 can continue in step 604 by obtaining reimbursement information for the patient-specific implants based on patient pathology data. The reimbursement information can include, without limitation, patient insurance information, treatment codes, diagnosis codes, supply item codes, procedure codes, or other information. The method 600 can determine applicable codes, such as primary or diagnosis codes, supply item codes, implant or device codes, or procedure codes, or the like. The reimbursement database (e.g., database 151 of FIG. 1) can store kit reimbursement information selecting or manufacturing kits, which can include one or more implants, instruments, instructions for use, and reusable and disposable components. In some embodiments, the method 600 can identify a surgical procedure to be performed and then retrieve requirements for reimbursement from the database. Systems (e.g., system 100 of FIG. 1) can synchronize the surgical plan with the reimbursement requirements to generate patient-specific surgical kits meeting the reimbursement requirements or including reimbursable items.

Step 604 can also include obtaining information for multi-kit procedures. For example, a decompression kit can be used to perform decompression procedures and additional kits can be used to implant devices. Systems can determine compatible kits that are reimbursable under the requirements for reimbursement. Additionally, coverage provided by insurance and reimbursement codes (e.g., reimbursement codes stored in the database 151, such as the reimbursement codes discussed in connection with FIG. 10B) can be matched with kits and/or kit components. Some procedures can include usage of a combination of standard kits and patient-specific kits for treatment flexibility.

The method 600 can continue in step 606 by associating reimbursement code(s) to items/kits/devices based on the reimbursement information. Patient pre-operative data can be analyzed to determine one or more primary or second diagnoses of the patient meeting at least one of a diagnosis code, a procedure code, a supply item code, or other code or parameter. A payment request can be submitted under identified code(s). The payment request can include ancillary payment/reimbursement, such as new technology add-on payment, diagnosis-related group payment, or other treatment-related payments.

The method 600 can include associating reimbursement codes (reimbursement codes discussed in connection with FIG. 10B) to treatments/patient-specific implants. These associations can be displayed in a plan or treatment report, as discussed in connection with FIGS. 10A and 10B. A user can review planned reimbursement plans prior to beginning the manufacturing process such that pre-operative changes to be implemented prior to beginning manufacturing of implants and other items for treatment. Moreover, the system can retrieve regulatory information and reimbursement information in real-time to ensure that the latest regulatory and reimbursement information is considered.

In step 608, the method 600 can include receiving user input via the interactive surgical plan. The user input can include approval of or modification to a planned or predicted pathology and for medical reimbursement under the at least one medical reimbursement code. The user input can be provided via, for example, the input elements 1054 of FIG. 10A, input 901 of FIG. 11, and other user input elements disclosed herein.

FIG. 6B is a flow diagram illustrating a method 620 for managing regulatory requirements, according to embodiments of the present technology. Steps of the method 620 can be implemented using treatment plans discussed in connection with FIGS. 10A-11. The method 620 can begin in step 622 by obtaining one or more images of a patient. In some embodiments, the step 622 includes selecting reference patient data sets each including one or more patient images, insurance information, and one or more medical reimbursement codes.

In step 624, a corrected patient pathology is designed using a surgical planning and reimbursement platform (e.g., SPR platform 109 of FIG. 1). The surgical planning and reimbursement platform can be programmed to design patient-specific implants qualifying for at least one medical reimbursement code. In some embodiments, method includes categorizing a pathology and then selecting the treatment based on the categorization. The pathology categorization can be matched with coding data (e.g., coding descriptions discussed in connection with FIG. 10B) to facilitate pathology categorization. The categorization can be based on thresholding pre-operative measurements, machine learning algorithms, etc. For example, the categorizations for spinal conditions can include spinal curvature deformity, nerve compression, and/or degenerative disc disease based on user input, regulatory indications, medical literature, or the like. One example categorization can be based on an ODI above a certain score, such as 30, 40, or 50. The system can store such regulatory indications in the database 151. Those regulatory indications, or other indications, can be retrieved and analyzed to identify potential categorizations for spinal conditions based on those indications. This allows synchronization between pathology categorizations and regulatory indications.

At step 626, regulatory design constraints can be obtained for the corrected pathology. The regulatory design constraints can include country-specific regulations, agency-specific regulations, governmental regulations, indications (e.g., threshold ODI, degenerative disc disease, etc.), designations (e.g., Breakthrough Device Designation), etc. The regulatory indication values can be ODI score (e.g., an ODI>40, ODI>30, etc.), threshold pre-operative measurements, etc. In some embodiments, the regulatory design constraints can include government agency requirements for approved implants. The example regulatory design constraints can include, without limitation, implant footprints (e.g., sizes, dimensions, etc.), materials, manufacturing constraints, sterilization procedures, or the like. The regulatory indication values can be based on the regulatory approval process. For example, the regulatory indication values can be published by a government agency, such as the FDA. The database 151 can automatically retrieve the regulatory indication values for treatments.

At step 628, virtual implants are fit to a virtual model representing corrected pathology. Simulations can be performed to confirm the likelihood of treatment outcomes based on the implant designs. In some design protocols, a type of patient-specific implant can be selected. The types can include, for example, interbody implant devices, artificial discs, single level spinal fusion system, multi-level single level spinal fusion system, joint replacements (e.g., artificial discs), hip implants, or other implants disclosed herein. In one example design process, anatomical elements of a patient's spine can be measured. The system can then select an implant footprint from a set of approved implant footprints (e.g., FDA approved implant footprints). For example, a footprint can be an interbody footprint for mating with vertebral bodies. The system can then virtually place the implant footprint along one vertebral endplate and can fill a negative space between opposing vertebral endplates. In this manner, an implant having a regulatory approved interbody footprint is designed to fit the patient's anatomy. The implant design process can be selected based on the pathology correction to be achieved.

At step 630, the implant(s) can be manufactured by a manufacturing system (e.g., manufacturing system 124 of FIG. 1). The manufacturing instructions for automated manufacturing can be generated based on the implant design step 628, thereby ensuring implant(s) meet with regulatory requirements.

At step 632, the manufacturing system 124 can check the manufacturing of the physical implants using, for example, an imager (e.g., analyzer or imager 131 of FIG. 1). In some checking procedures, images of the implant can be analyzed to, for example, measure the implant, inspect surface features of implants, or other characteristics of the implant. Additionally or alternatively, the implant can be measured, weighed, subjected to non-destructive testing, or other protocols. Other checking techniques can be performed. Additionally, the implant can be analyzed to confirm that it is meets reimbursement requirements. If third-party payor payment or reimbursement requires that the implant be included with a kit, the implant can be assembled with other items to product a customized kit. Step 632 can also be performed by healthcare providers, hospitals, surgical teams, and/or physicians.

Machine learning algorithms can be used perform one or more steps methods 600 of FIG. 6A and 620 of FIG. 6B. For example, the SPR platform 109 of FIG. 1 can include a machine-learning model trained using the selected reference patient data sets. Patient images can be inputted into the trained machine-learning model to generate the corrected patient pathology that meets, for example, insurance requirements, one or more reimbursement parameters, or the like. The machine learning model can be selected based on design goals, such as optimized patient outcomes, reimbursement requirements, patient costs.

FIGS. 7A-13 further illustrate select aspects of providing patient-specific medical care, e.g., in accordance with the method 500. For example, FIG. 7A-7D illustrate an example of a patient data set 700 (e.g., as received in step 502 of the method 500). The patient data set 700 can include any of the information previously described with respect to the patient data set. For example, the patient data set 700 includes patient information 701 (e.g., patient identification no., patient MRN, patient name, sex, age, body mass index (BMI), surgery date, surgeon, etc., shown in FIGS. 7A and 7B), diagnostic information 702 (e.g., Oswestry Disability Index (ODI), VAS-back score, VAS-leg score, Pre-operative pelvic incidence, pre-operative lumbar lordosis, pre-operative PI-LL angel, pre-operative lumbar coronal cobb, etc., shown in FIGS. 7B and 7C), and image data 703 (x-ray, CT, MRI, etc., shown in FIG. 7D). In the illustrated embodiment, the patient data set 700 is collected by a healthcare provider (e.g., a surgeon, a nurse, etc.) using a digital and/or fillable report that can be accessed using a computing device. In some embodiments, the patient data set 700 can be automatically or at least partially automatically generated based on digital medical records of the patient. Regardless, once collected, the patient data set 700 can be transmitted to the computing system configured to generate the surgical plan for the patient.

FIGS. 8A and 8B illustrate an example of a virtual model 800 of a patient's native anatomical configuration (e.g., as created in step 503 of the method 500). In particular, FIG. 8A is an enlarged view of the virtual model 800 of the patient's native anatomy and shows the patient's native anatomy of their lower spinal cord region. The virtual model 800 is a three-dimensional visual representation of the patient's native anatomy. In the illustrated embodiment, the virtual model includes a portion of the spinal column extending from the sacrum to the L4 vertebral level. Of course, the virtual model can include other regions of the patient's spinal column, including cervical vertebrae, thoracic vertebrae, lumbar vertebrae, and the sacrum. The illustrated virtual model 800 only includes bony structures of the patient's anatomy, but in other embodiments may include additional structures, such as cartilage, soft tissue, vascular tissue, nervous tissue, etc.

FIG. 8B illustrates a virtual model display 850 (referred to herein as the “display 850”) showing different views of the virtual model 800. The virtual model display 850 includes a three-dimensional view of the virtual model 800, one or more coronal cross-section(s) 802 of the virtual model 800, one or more axial cross section(s) 804 of the virtual model 800, and/or one or more sagittal cross-section(s) 806 of the virtual model 800. Of course, other views are possible and can be included on the virtual model display 850. In some embodiments, the virtual model 800 may be interactive such that a user can manipulate the orientation or view of the virtual model 800 (e.g., rotate), change the depth of the displayed cross-sections, select and isolate specific bony structures, or the like.

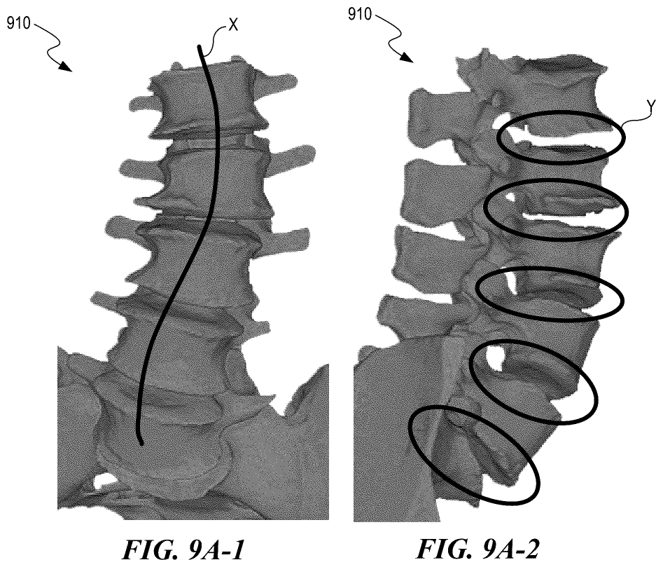

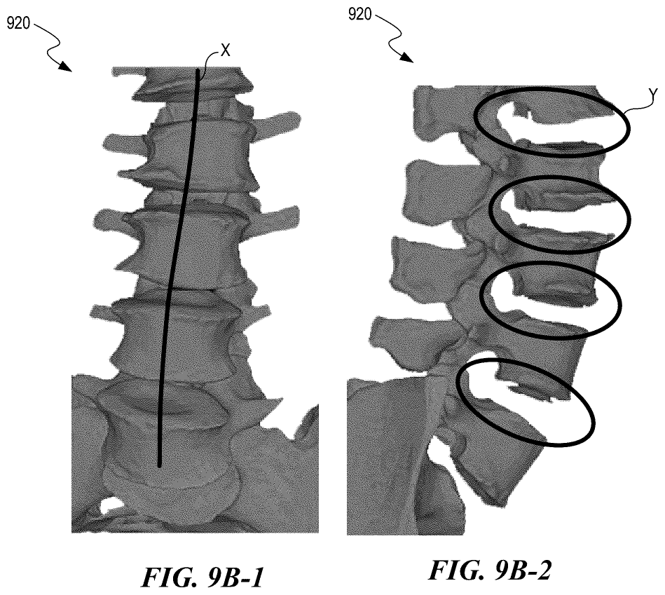

FIGS. 9A-1-9B-2 demonstrate an example of a virtual model of a patient's native anatomical configuration (e.g., as created in step 503 of the method 500) and a virtual model of the patient's corrected anatomical configuration (e.g., as created in step 504 of the method 500). In particular, FIGS. 9A-1 and 9A-2 are anterior and lateral views, respectively, of a virtual model 910 showing a native anatomical configuration of a patient, and FIGS. 9B-1 and 9B-2 are anterior and lateral views, respectively, of a virtual model 920 showing the corrected anatomical configuration for the same patient. Referring first to FIG. 9A-1, the anterior view of the virtual model 910 illustrates the patient has abnormal curvature (e.g., scoliosis) of their spinal column. This is marked by line X, which follows a rostral-caudal axis of the spinal column. Referring next to FIG. 9A-1, the lateral view of the virtual model 910 illustrates the patient has collapsed discs or decreased spacing between adjacent vertebral endplates, marked by ovals Y. FIGS. 9B-1 and 9B-2 illustrate the corrected virtual model 920 accounting for the abnormal anatomical configurations shown in FIGS. 9A-1 and 9A-2. For example, FIG. 9B-1, which is an anterior view of the virtual model 920, illustrates the patient's spinal column having corrected alignment (e.g., the abnormal curvature has been reduced). This correction is shown by line X, which also follows a rostral-caudal axis of the spinal column. FIG. 9B-2, which is a lateral view of the virtual model 920, illustrates the patient's spinal column having restored disc height (e.g., increased spacing between adjacent vertebral endplates), also marked by ovals Y. The lines X and the ovals Y are provided in FIGS. 9A-1-9B-2 to more clearly demonstrate the correction between the virtual models 910 and 920, and are not necessarily included on the virtual models generated in accordance with the present technology.

FIG. 10 illustrates an example of a surgical plan 1000 (e.g., as generated in step 506 of the method 500). The surgical plan 1000 can include pre-operative patient metrics or measurements 1002, predicted post-operative patient metrics 1004, one or more patient images (e.g., the patient images 703 received as part of the patient data set), the virtual model 910 (which can be the model itself or one or more images derived from the model) of the patient's native anatomical configuration (e.g., pre-operative patient anatomy), and/or the virtual model 920 (which can be the model itself or one or more images derived from the model) of the patient's corrected anatomical configuration (e.g., predicted post-operative patient anatomy). The pre-operative patient metrics or measurements 1002 can include, without limitation, lumbar lordosis, Cobb angle(s), pelvic incidence, disc height, coronal offset distance, segment flexibility, LL-PI is greater than predetermined degrees (e.g., 5 degrees, 10 degrees, etc.), LL-PI mismatch (e.g., age-adjusted), sagittal vertical axis offset distance, coronal offset distance, coronal angle, bone quality, rotational displacement.

The virtual model 920 of the predicted post-operative patient anatomy can optionally include one or more implants 1012 shown as implanted in the patient's spinal cord region to demonstrate how patient anatomy will look following the surgery. Although four implants 1012 are shown in the virtual model 920, the surgical plan 1000 may include more or fewer implants 1012, including one, two, three, five, six, seven, eight, or more implants 1012.

The surgical plan 1000 can include additional information beyond what is illustrated in FIG. 10. For example, the surgical plan 1000 may include pre-operative instructions, operative instructions, and/or post-operative instructions. Operative instructions can include one or more specific procedures to be performed (e.g., PLIF, ALIF, TLIF, LLIF, DLIF, XLIF, etc.) and/or one or more specific targets of the operation (e.g., fusion of vertebral levels L1-L4, anchoring screw to be inserted in lateral surface of L4, etc.). Although the surgical plan 1000 is demonstrated in FIG. 10 as a visual report, the surgical plan 1000 can also be encoded in computer-executable instructions that, when executed by a processor connected to a computing device, cause the surgical plan 1000 to be displayed by the computing device. In some embodiments, the surgical plan 1000 may also include machine-readable operative instructions for carrying out the surgical plan. For example, the surgical plan can include operative instructions for a robotic surgical platform to carry out one or more steps of the surgical plan 1000.

FIG. 10A shows the surgical plan 1000 including interactive RCR treatment plan 1016 having, for example, regulatory information 1024, reimbursement information 1034, plan comparisons 1044, user input element(s) 1054, and verification data 1062. The regulatory information 1024 can include one or more regulatory indication values associated with the reimbursement information 1034. The user can input modifications via the user input elements 1054 to dynamically update at least one of regulatory approval or reimbursement information 1024, 1034. For example, the user can input modifications to user adjustable values (e.g., planned metrics 1004, position of anatomical elements in models 920, etc.). The system can automatically update other information in the plan 1000. This allows a user to view changes to regulatory requirements, reimbursements, and other information based on planned treatment modifications. In some embodiments, the user can modify one or more of the planned metrics 1004. The surgical plan 1000 is then updated to display the corresponding anatomical correction 920 and reimbursement information associated for achieving those modified planned metrics 1004. The plan comparisons 1044 can include pre- and post-modified anatomical corrections, post-operative long-term outcome comparisons, cost comparisons (e.g., cost to healthcare provider, patient, etc.), treatment risk comparisons, or the like. The cost comparisons can be obscured (e.g., unviewable by a physician), or not displayed, until treatment has been selected. Features of the surgical plan 1000 are discussed below.

The reimbursement information 1034 can include medical reimbursement codes for treatment plans. The reimbursement information 1034 can include the medical imbursement processor information, such as private insurance processing information, governmental insurance processor (e.g., Medicare or Medicaid), etc. The reimbursement information 1034 can also include one or more primary or second diagnoses, treatment type or description of treatment, technology codes (e.g., new technology add-on reimbursements), diagnoses related groups reimbursements, or the like. The reimbursement information 1034 can include one or more selectable links for accessing code data (e.g., code data of FIG. 10B) associated with the pre-operative metrics, model 910, and/or images 703. The codes can be overlaid or inserted onto the models 910 and/or pre-operative images 703. Details of recommended coding are discussed in connection with FIG. 10B. The verification 1062 can confirm coding is accurate.

The plan comparisons 1044 can compare two or more surgical plans and include, without limitation, reimbursement or cost comparisons, patient metrics comparisons (e.g., plan metrics similar to metrics 1004), etc. The reimbursement or cost comparisons can include patient costs, payments to healthcare provider, imbursements, or the like based on payment schemes, including new technology add-on payment, diagnosis-related group payment, or other treatment-related payments. The verification 1062 can confirm whether the planned treatment is approved by a government regulatory agency. This allows a user to visually evaluate planned outcomes based on costs to patients, healthcare provider costs, and other financial considerations.

FIG. 10B shows codes for a treatment plan or patient. In the illustrated embodiment, the codes include primary ICD-10-CM primary and secondary diagnostic codes and primary codes associated with spinal conditions. A box or window 1037 can include recommended codes. A user can review the recommended codes in view of the analytics and other information in the treatment plan, as discussed in connection with FIG. 10A. The information in window 1037 of FIG. 10B can be included in or linked to the window 1034 of FIG. 10A.

If the codes are acceptable, the user can approve or reject the codes. For example, the user can select the approve button 1041 to approve the recommended codes. The recommended codes can then be included in a reimbursement report generated for reimbursement submission. If the codes are unacceptable, user can select the reject button 1043. The system can receive input from the user and then generate new recommendations. For example, the user can upload new images (e.g., images 703 of FIG. 10A) for further analysis. The system can analyze the newly uploaded images to generate new analytics. Based on the new analytics, the system can reevaluate the recommended codes. If the system is unable to arrive at a sufficient confidence level for coding, the system can recommend obtaining additional patient data (e.g., patient images, questionnaire feedback, physician feedback) to obtain a sufficient amount of data for generating recommended coding at the threshold confidence level.

The condition of the spine can be analyzed and matched with codes using, for example, machine learning algorithms. This process can be repeated any number of times until the recommended codes are approved via the button 1041. Advantageously, the user can select the recommended code to view the basis for the recommendation. For example, the user can select the code Q67.5. The report can then be modified to highlight and label the measurements, physician notes, patient elements and patient images, anatomical elements in the virtual models, or other features serving the basis for the recommendation (e.g., features indicating congenital deformity of spine). This allows a user to evaluate coding prior to approving a surgical treatment plan. The system can also recommend one or more diagnostic procedures to be performed to provide sufficient information for additional coding analysis.

FIG. 11 provides a series of images illustrating an example of a patient surgical plan report 1100 that includes the surgical plan 1000 and that may be transmitted to a surgeon for review and approval (e.g., as transmitted in step 508 of the method 500). The surgical plan report 1100 can include a multi-page report detailing aspects of the surgical plan 1000. For example, the multi-page report may include a first page 1101 demonstrating an overview of the surgical plan 1000 (e.g., as shown in FIG. 10), a second page 1102 illustrating patient images (e.g., such as the patient images 703 received in step 502 and shown in FIG. 7D), a third page 1103 illustrating an enlarged view of the virtual model of the corrected anatomical configuration (e.g., the virtual model 920 shown in FIG. 9), and a fourth page 1104 prompting the surgeon to either approve or reject the surgical plan 900 via a user input element 901 (e.g., one or more buttons, a drop down menu, etc.). The surgical plan report 1100 can include one or more pre-operative metrics for pre-determined indications.

Page two 1102 can include pre-operative metrics 1109 determined based on the patient images 1113. The pre-operative metrics 1109 can be used to perform a reimbursement analysis, including whether a procedure, kit, instrument, implants, or other treatment-related item or step will qualify for payment or reimbursement. In some embodiments, planned metrics 1118 (page 1101) can be used to validate a predicted outcome for the pre-determined indications will qualify for payment or reimbursement.

Page two 1102 can also include reimbursement data 123 and regulatory data 127. The reimbursement data 123 can include the data discussed in connection with FIG. 10B. The output (e.g., recommended codes) can be labeled in the illustrated images 703. The pre-operative metrics 1109 correlated to the coding can be bolded or otherwise identified. This allows a user to simultaneously view reimbursement information and physiology associated with those reimbursements. The regulatory data 127 can include images of virtual models with anatomical features and regulatory compliant implants. The planned anatomical model (e.g., virtual anatomical model 920 of FIG. 10A) can have implants with regulatory approved configurations. The physician can therefore have confidence that the implants and planned outcome is based on regulatory approved technology.

In some embodiments, the system can measure the anatomical features and generate virtual models. The system can then generate the regulatory compliant implants that fit the model. If the physician modifies the model or implants resulting in a non-regulatory compliant treatment or implant, the system can generate an alert indicating that regulatory compliance has not been maintained. Advantageously, page 1102 allows a user to simultaneously view patient images, anatomical planned models, planned pathologies based on regulatory compliance, reimbursement data, and regulatory data. Moreover, correlations between various elements of different data sets can be identified to enable a viewer to understand the interrelationships.

Of course, additional information about the surgical plan can be presented with the report 1100 in the same or different formats. In some embodiments, if the surgeon rejects the surgical plan 1000, the surgeon can be prompted to provide feedback regarding the aspects of the surgical plan 1000 the surgeon would like adjusted.

The patient surgical plan report 1100 can be presented to the surgeon on a digital display of a computing device (e.g., the client computing device 102 shown in FIG. 1). In some embodiments, the report 1100 is interactive and the surgeon can manipulate various aspects of the report 1100 (e.g., adjust views of the virtual model, zoom-in, zoom-out, annotate, etc.). However, even if the report 1100 is interactive, the surgeon generally cannot directly change the surgical plan 1000. Rather, the surgeon may provide feedback and suggested changes to the surgical plan 1000, which can be sent back to the computing system that generated the surgical plan 1000 for analysis and refinement.

FIG. 12A illustrates an example of a patient-specific implant 1200 (e.g., as designed in step 516 and manufactured in step 518 of the method 500), and FIG. 12B illustrates the implant 1200 implanted in the patient. The implant 1200 can be any orthopedic or other implant specifically designed to induce the patient's body to conform to the previously identified corrected anatomical configuration. The implant 1220 can be based on a design generated by mapping a negative space between segmented anatomic elements of a corrected pathology. The negative space is then filled with a virtual implant. In imbursement-constrained embodiments, the configuration of the negative space can be selected based on one or more parameters for the medical reimbursable virtual implant. For example, the implant 1200 can be a cervical fusion implant, a lumbar fusion implant, an artificial disc, an expandable intervertebral cage, or other implant disclosed herein.

For example, system 100 of FIG. 1 can obtaining insurance information for the patient from the database 151. The system 100 can then retrieve one or more design parameters from a database 151 based on the obtained insurance information. The treatment model 181 can then design the patient-specific implant 1202 using the retrieved design parameter(s). The design parameters can be a configuration (e.g., an implant footprint shown in dashed line in FIG. 12A) for devices approved for use by regulatory agency or governmental body, payment requirement, imbursement requirement, etc. Prior to manufacturing the implant 1202, the system can notify the user of the at least one medical reimbursement code for user review and approval.

In the illustrated embodiment, the implant 1200 is a vertebral interbody device having a first (e.g., upper) surface 1206 configured to engage an inferior endplate surface of a superior vertebral body and a second (e.g., lower) surface 1204 configured to engage a superior endplate surface of an inferior vertebral body. The first surface 1206 can have a patient-specific topography designed to match (e.g., mate with) the topography of the inferior endplate surface of the superior vertebral body to form a generally gapless interface therebetween. Likewise, the second surface 1204 can have a patient-specific topography designed to match or mate with the topography of the superior endplate surface of the inferior vertebral body to form a generally gapless interface therebetween. The implant 1200 may also include a recess or other feature configured to promote bony ingrowth. Because the implant 1200 is patient-specific and designed to induce a geometric change in the patient, the implant 1200 is not necessarily symmetric, and is often asymmetric. For example, in the illustrated embodiment, the implant 1200 has a non-uniform thickness such that a plane defined by the first surface 1206 is not parallel to a central longitudinal axis A of the implant 1200. Of course, because the implants described herein, including the implant 1200, are patient-specific, the present technology is not limited to any particular implant design or characteristic. Additional features of patient-specific implants that can be designed and manufactured in accordance with the present technology are described in U.S. patent application Ser. Nos. 16/987,113 and 17/100,396, the disclosures of which are incorporated by reference herein in their entireties.

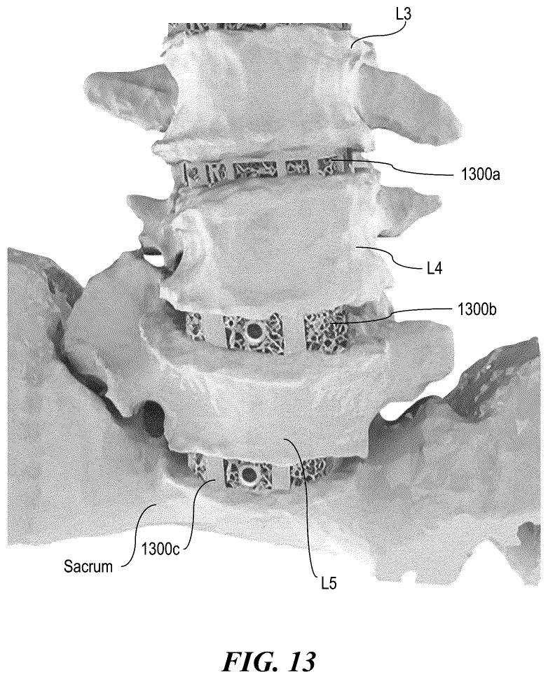

The patient-specific medical procedures described herein can involve implanting more than one patient-specific implant into the patient to achieve the corrected anatomical configuration (e.g., a multi-site procedure). FIG. 13, for example, illustrates a lower spinal cord region having three patient specific implants 1300a-1300c implanted at different vertebral levels. More specifically, a first implant 1300a is implanted between the L3 and L4 vertebral bodies, a second implant 1300b is implanted between the L4 and L5 vertebral bodies, and a third implant 1300c is implanted between the L5 vertebral body and the sacrum. Together, the implants 1300a-c can cause the patient's spinal cord region to assume the previously identified corrected anatomical configuration (e.g., transforming the patient's anatomy from its pre-operative diseased configuration to the post-operative optimized configuration). In some embodiments, more or fewer implants are used to achieve the corrected anatomical configuration. For example, in some embodiments one, two, four, five, six, seven, eight, or more implants are used to achieve the corrected anatomical configuration. In embodiments involving more than one implant, the implants do not necessarily have the same shape, size, or function. In fact, the multiple implants will often have different geometries and topographies to correspond to the target vertebral level at which they will be implanted. As also shown in FIG. 13, the patient-specific medical procedures described herein can involve treating the patient at multiple target regions (e.g., multiple vertebral levels).

In addition to designing patient-specific medical care based off reference patient data sets, the systems and methods of the present technology may also design patient-specific medical care based off disease progression for a particular patient. In some embodiments, the present technology therefore includes software modules (e.g., machine learning models or other algorithms) that can be used to analyze, predict, and/or model disease progression for a particular patient. The machine learning models can be trained based off a plurality of reference patient data sets that includes, in addition to the patient data described with respect to FIG. 1, disease progression metrics for each of the reference patients. The progression metrics can include measurements for disease metrics over a period of time. Suitable metrics may include spinopelvic parameters (e.g., lumbar lordosis, pelvic tilt, sagittal vertical axis (SVA), cobb angel, coronal offset, etc.), disability scores, functional ability scores, flexibility scores, VAS pain scores, or the like. The progression of the metrics for each reference patient can be correlated to other patient information for the specific reference patient (e.g., age, sex, height, weight, activity level, diet, etc.).

In some embodiments, the present technology includes a disease progression module that includes an algorithm, machine learning model, or other software analytical tool for predicting disease progression in a particular patient. The disease progression module can be trained based on reference patient data sets that includes patient information (e.g., age, sex, height, weight, activity level, diet) and disease metrics (e.g., diagnosis, spinopelvic parameters such as lumbar lordosis, pelvic tilt, sagittal vertical axis, cobb angel, coronal offset, etc., disability scores, functional ability scores, flexibility scores, VAS pain scores, etc.). The disease metrics can include values over a period of time. For example, the reference patient data may include values of disease metrics on a daily, weekly, monthly, bi-monthly, yearly, or other basis. By measuring the metrics over a period of time, changes in the values of the metrics can be tracked as an estimate of disease progression and correlated to other patient data.

In some embodiments, the disease progression module can therefore estimate the rate of disease progression for a particular patient. The progression may be estimated by providing estimated changes in one or more disease metrics over a period of time (e.g., X % increase in a disease metric per year). The rate can be constant (e.g., 5% increase in pelvic tilt per year) or variable (e.g., 5% increase in pelvic tilt for a first year, 10% increase in pelvic tilt for a second year, etc.). In some embodiments, the estimated rate of progression can be transmitted to a surgeon or other healthcare provider, who can review and update the estimate, if necessary.

As a non-limiting example, a particular patient who is a fifty-five-year-old male may have a SVA value of 6 mm. The disease progression module can analyze patient reference data sets to identify disease progression for individual reference patients have one or more similarities with the particular patient (e.g., individual patients of the reference patients who have an SVA value of about 6 mm and are approximately the same age, weight, height, and/or sex of the patient). Based on this analysis, the disease progression module can predict the rate of disease progression if no surgical intervention occurs (e.g., the patient's VAS pain scores may increase 5%, 10%, or 15% annually if no surgical intervention occurs, the SVA value may continue to increase by 5% annually if no surgical intervention occurs, etc.).

The systems and methods described herein can also generate models/simulations based on the estimated rates of disease progression, thereby modeling different outcomes over a desired period of times. Additionally, the models/simulations can account for any number of additional diseases or condition to predict the patient's overall health, mobility, or the like. These additional diseases or conditions can, in combination with other patient health factors (e.g., height, weight, age, activity level, etc.) be used to generate a patient health score reflecting the overall health of the patient. The patient health score can be displayed for surgeon review and/or incorporated into the estimation of disease progression. Accordingly, the present technology can generate one or more virtual simulations of the predicted disease progression to demonstrate how the patient's anatomy is predicted to change over time. Physician input can be used to generate or modify the virtual simulation(s). The present technology can generate one or more post-treatment virtual simulations based on the received physician input for review by the healthcare provider, patient, etc.

In some embodiments, the present technology can also predict, model, and/or simulate disease progression based on one or more potential surgical interventions. For example, the disease progression module may simulate what a patient's anatomy may look like 1, 2, 5, or 10 years post-surgery for several surgical intervention options. The simulations may also incorporate non-surgical factors, such as patient age, height, weight, sex, activity level, other health conditions, or the like, as previously described. Based on these simulations, the system and/or a surgeon can select which surgical intervention is best suited for long-term efficacy. These simulations can also be used to determine patient-specific corrections that compensate for the projected diseases progression.

Accordingly, in some embodiments, multiple disease progression models (e.g., two, three, four, five, six, or more) are simulated to provide disease progression data for several different surgical intervention options or other scenarios. For example, the disease progression module can generate models that predict post-surgical disease progression for each of three different surgical interventions. A surgeon or other healthcare provider can review the disease progression models and, based on the review, select which of the three surgical intervention options is likely to provide the patient with the best long-term outcome. Of course, selecting the optimal intervention can also be fully or semi-automated, as described herein.

Based off of the modeled disease progression, the systems and methods described herein can also (i) identify the optimal time for surgical intervention, and/or (ii) identify the optimal type of surgical procedure for the patient. In some embodiments, the present technology therefore includes an intervention timing module that includes an algorithm, machine learning model, or other software analytical tool for determining the optimal time for surgical intervention in a particular patient. This can be done, for example, by analyzing patient reference data that includes (i) pre-operative disease progression metrics for individual reference patients, (ii) disease metrics at the time of surgical intervention for individual reference patients, (iii) post-operative disease progression metrics for individual reference patients, and/or (iv) scored surgical outcomes for individual reference patients. The intervention timing module can compare the disease metrics for a particular patient to the reference patient data sets to determine, for similar patients, the point of disease progression at which surgical intervention produced the most favorable outcomes.

As a non-limiting example, the reference patient data sets may include data associated with reference patients' sagittal vertical axis. The data can include (i) sagittal vertical axis values for individual patients over a period of time before surgical intervention (e.g., how fast and to what degree the sagittal vertical axis value changed), (ii) sagittal vertical axis of the individual patients at the time of surgical intervention, (iii) the change in sagittal vertical axis after surgical intervention, and (iv) the degree to which the surgical intervention was successful (e.g., based on pain, quality of life, or other factors). Based on the foregoing data, the intervention timing module can, based on a particular patient's sagittal vertical axis value, identify at which point surgical intervention will have the highest likelihood of producing the most favorable outcome. Of course, the foregoing metric is provided by way of example only, and the intervention timing module can incorporate other metrics (e.g., lumbar lordosis, pelvic tilt, sagittal vertical axis, cobb angel, coronal offset, disability scores, functional ability scores, flexibility scores, VAS pain scores) instead of or in combination with sagittal vertical axis to predict the time at which surgical intervention has the highest probability of providing a favorable outcome for the particular patient.

The intervention timing module may also incorporate one or more mathematical rules based on value thresholds for various disease metrics. For example, the intervention timing module may indicate surgical intervention is necessary if one or more disease metrics exceed a predetermined threshold or meet some other criteria. Representative thresholds that indicate surgical intervention may be necessary include SVA values greater than 7 mm, a mismatch between lumbar lordosis and pelvic incidence greater than 10 degrees, a cobb angle of greater than 10 degrees, and/or a combination of cobb angle and LL/PI mismatch greater than 20 degrees. Of course, other threshold values and metrics can be used; the foregoing are provided as examples only and in no way limit the present disclosure. In some embodiments, the foregoing rules can be tailored to specific patient populations (e.g., for males over 50 years of age, an SVA value greater than 7 mm indicates the need for surgical intervention). If a particular patient does not exceed the thresholds indicating surgical intervention is recommended, the intervention timing module may provide an estimate for when the patient's metrics will exceed one or more thresholds, thereby providing the patient with an estimate of when surgical intervention may become recommended.

The present technology may also include a treatment planning module that can identify the optimal type of surgical procedure for the patient based on the disease progression of the patient. The treatment planning module can be an algorithm, machine learning model, or other software analytical tool trained or otherwise based on a plurality of reference patient data sets, as previously described. The treatment planning module may also incorporate one or more mathematical rules for identifying surgical procedures. As a non-limiting example, if a LL/PI mismatch is between 10 and 20 degrees, the treatment planning module may recommend an anterior fusion surgery, but if the LL/PI mismatch is greater than 20 degrees, the treatment planning module may recommend both anterior and posterior fusion surgery. As another non-limiting example, if a SVA value is between 7 mm and 15 mm, the treatment planning module may recommend posterior fusion surgery, but if the SVA is above 15 mm, the treatment planning module may recommend both posterior fusion surgery and anterior fusion surgery. Of course, other rules can be used; the foregoing are provided as examples only and in no way limit the present disclosure.

Without being bound by theory, incorporating disease progression modeling into the patient-specific medical procedures described herein may even further increase the effectiveness of the procedures. For example, in many cases it may be disadvantageous operate after a patient's disease progresses to an irreversible or unstable state. However, it may also be disadvantageous to operate too early, before the patient's disease is causing symptoms and/or if the patient's disease may not progress further. The disease progression module and/or the intervention timing module can therefore help identify the window of time during which surgical intervention in a particular patient has the highest probability of providing a favorable outcome for the patient.

As one skilled in the art will appreciate, any of the software modules described previously may be combined into a single software module for performing the operations described herein. Likewise, the software modules can be distributed across any combination of the computing systems and devices described herein, and are not limited to the express arrangements described herein. Accordingly, any of the operations described herein can be performed by any of the computing devices or systems described herein, unless expressly noted otherwise.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

### Examples

The present technology is illustrated, for example, according to various aspects described below. Various examples of aspects of the present technology are described as numbered examples (1, 2, 3, etc.) for convenience. These are provided as examples and do not limit the present technology. It is noted that any of the dependent examples can be combined in any suitable manner, and placed into a respective independent example. The other examples can be presented in a similar manner.

1. A computer-implemented method for designing patient-specific implant based on a patient-specific interactive surgical plan, the method comprising:
displaying, via a display, the patient-specific interactive surgical plan generated by a surgical planning platform based on one or more images of a patient,
   wherein the patient-specific interactive surgical plan includes a user input element for modifying and/or approving the patient-specific interactive surgical plan, wherein the patient-specific interactive surgical plan includes a viewable planned post-operative pathology for the patient,
obtaining reimbursement information for one or more patient-specific implants based on diagnosis data, procedural data, or pathology data of the patient in the patient-specific interactive surgical plan;
assigning at least one medical reimbursement code to the one or more patient-specific implants based on the reimbursement information; and
receiving, via the patient-specific interactive surgical plan, approval of or modification to the planned post-operative pathology, wherein the surgical planning platform is configured to design the one or more patient-specific implants for achieving the approved planned post-operative pathology and for medical reimbursement under the at least one medical reimbursement code.

2. The computer-implemented method of example 1, wherein the surgical planning platform is configured to
store, via a regulatory and reimbursement manager, a first medical reimbursement code and a second medical reimbursement code in a medical code database;
generate a virtual three-dimensional model representing the patient's anatomy;
manipulate the virtual three-dimensional model to generate a first surgical plan that qualifies for reimbursement under the first medical reimbursement code in the medical code database; and
manipulate the virtual three-dimensional model to generate a second surgical plan that qualifies for reimbursement under the second medical reimbursement code in the medical code database.

3. The computer-implemented method of any of examples 1-2, further comprising:
displaying a comparison of the first and second surgical plans, wherein the comparison includes
   a reimbursement comparison between the first and second surgical plans; and
   a predicted patient outcome comparison between the first and second surgical plans.

4. The computer-implemented method of any of examples 1-3, wherein the patient-specific interactive surgical plan includes one or more regulatory indication values associated with a treatment provided by the one or more patient-specific implants.

5. The computer-implemented method of any of examples 1-4, further comprising:
displaying a user inputted modification to the patient-specific interactive surgical plan; and
dynamically updating at least one of regulatory approval or reimbursement information, wherein the updating is based on the user inputted modification.

6. The computer-implemented method of any of examples 1-5, further comprising displaying
one or more user adjustable values,
an anatomical correction based on the one or more user adjustable values, and
reimbursement information associated with treatment for achieving the anatomical correction.

7. The computer-implemented method of any of examples 1-6, further comprising:
verifying accuracy of matching of the at least one medical reimbursement code to the one or more patient-specific implants based on at least one predetermined processing rule prior to transmitting a reimbursement request for the treatment to a medical imbursement processor;
displaying verification that treatment using the one or more patient-specific implants is approved by a government regulatory agency; and
displaying pricing based on a new technology add-on payment and/or diagnosis-related group payment.

8. The computer-implemented method of any of examples 1-7, further comprising:
obtaining one or more pre-operative metrics for pre-determined indications; and
obtaining one or more post-operative metrics to validate a target outcome for the pre-determined indications.

9. The computer-implemented method of any of examples 1-8, wherein the obtained reimbursement information includes at least one of treatment type or description of treatment.

10. The computer-implemented method of any of examples 1-9, further comprising:
coding the one or more patient-specific devices for multiple coded reimbursements,
   wherein the multiple coded reimbursements include:
   a new technology add-on reimbursement; and
   a diagnoses-related groups reimbursement.

11. A system comprising:
one or more processors; and
one or more memories storing instructions that, when executed by the one or more processors, cause the system to perform a process comprising:
   displaying, via a display, a patient-specific interactive surgical plan generated by a surgical planning platform based on one or more images of a patient,
      wherein the patient-specific interactive surgical plan includes a user input element for modifying and/or approving the patient-specific interactive surgical plan, wherein the patient-specific interactive surgical plan includes a viewable planned post-operative pathology for the patient;
   obtaining reimbursement information for one or more patient-specific implants based on pathology data of the patient in the patient-specific interactive surgical plan;
   assigning at least one medical reimbursement code to the one or more patient-specific implants based on the reimbursement information; and
   receiving, via the patient-specific interactive surgical plan, approval of or modification to the planned post-operative pathology, wherein the surgical planning platform is configured to design the one or more patient-specific implants for achieving the approved planned post-operative pathology and for medical reimbursement under the at least one medical reimbursement code.

12. A non-transitory computer-readable medium storing instructions that, when executed by a computing system, cause the computing system to perform operations comprising:
displaying, via a display, a patient-specific interactive surgical plan generated by a surgical planning platform based on one or more images of a patient,
wherein the patient-specific interactive surgical plan includes a user input element for modifying and/or approving the patient-specific interactive surgical plan, wherein the patient-specific interactive surgical plan includes a viewable planned post-operative pathology for the patient,
obtaining reimbursement information for one or more patient-specific implants based on pathology data of the patient in the patient-specific interactive surgical plan;
assigning at least one medical reimbursement code to the one or more patient-specific implants based on the reimbursement information; and
receiving, via the patient-specific interactive surgical plan, approval of or modification to the planned post-operative pathology, wherein the surgical planning platform is configured to design the one or more patient-specific implants for achieving the approved planned post-operative pathology and for medical reimbursement under the at least one medical reimbursement code.

13. A computer-implemented method comprising:
obtaining one or more images of a patient;
designing a corrected patient pathology based on the one or more images using a surgical planning and reimbursement platform, wherein the surgical planning and reimbursement platform is programmed to design implants qualifying for at least one medical reimbursement code;
generating, using the surgical planning and reimbursement platform, an interactive surgical plan that includes a viewable corrected patient pathology for modifying and/or approving the corrected patient pathology; and
designing, using the surgical planning and reimbursement platform, one or more patient-specific implants for achieving the approved corrected patient pathology and for medical reimbursement under the at least one medical reimbursement code.

14. The computer-implemented method of example 13, further comprising:
selecting reference patient data sets each including one or more patient images and one or more medical reimbursement codes;
training a machine-learning model using the selected reference patient data sets; and
inputting the one or more patient images of the patient into the trained machine-learning model to generate the corrected patient pathology that meets one or more reimbursement parameters.

15. The computer-implemented method of any of examples 13-14, further comprising:

verifying, using a regulatory and reimbursement manager, that the designed one or more patient-specific implants qualify for medical reimbursement under the at least one medical reimbursement code.

16. The computer-implemented method of any of examples 13-15, further comprising:
storing an implant design constraint associated with the at least one medical reimbursement code or regulatory approval; and
filling a negative space between anatomical elements of the corrected patient pathology to generate one or more virtual patient-specific implants meeting the implant design constraint.

17. The computer-implemented method of any of examples 13-16, wherein the implant design constraint is a footprint of a vertebral cage.

18. The computer-implemented method of any of examples 13-17, further comprising:
obtaining insurance information for the patient;
segmenting anatomy of interest in at least one of the one or more images;
isolating separate anatomic elements of the anatomy of interest; and
manipulating spatial relationships between the isolated anatomic elements based on the insurance information to
   map a negative space between the anatomic elements, and
   fill at least a portion of the negative space with a medical reimbursable virtual implant.

19. The computer-implemented method of any of examples 13-18, further comprising:
obtaining insurance information for the patient;
segmenting anatomy of interest in at least one of the one or more images; and
manipulating spatial relationships between the segmented anatomy based on the insurance information to design the corrected patient pathology.

20. The computer-implemented method of any of examples 13-19, wherein the corrected patient pathology is represented by isolated anatomic elements of the patient, wherein the method further comprises:
filling at least a portion of a negative space between two or more of the isolated anatomic elements with a virtual implant representing the at least one of the one or more patient-specific implants.

21. The computer-implemented method of any of examples 13-20, further comprising:
obtaining insurance information for the patient;
retrieving one or more design parameters from a reimbursement database based on the obtained insurance information, wherein the one or more design parameters are associated with medical reimbursement under the at least one medical reimbursement code; and
using the one or more design parameters to design the one or more patient-specific implants.

22. The computer-implemented method of any of examples 13-21, further comprising:
identifying the at least one medical reimbursement code based on the corrected patient pathology; and
notifying a user of the at least one medical reimbursement code.

23. The computer-implemented method of any of examples 13-22, further comprising:
creating a virtual 3D model of the one or more patient-specific implants to fit between anatomical elements of the corrected patient pathology;
converting the virtual 3D model into 3D fabrication data; and
manufacturing at least a portion of the one or more patient-specific implants based on the 3D fabrication data.

24. The computer-implemented method of any of examples 13-23, further comprising:
obtaining reimbursement verification data based on insurance or coverage information for the patient; and
verifying the one or more patient-specific implants qualify for medical reimbursement under the at least one medical reimbursement code based on the reimbursement verification data.

25. The computer-implemented method of any of examples 13-24, further comprising:
comparing one or more pre-operative metrics to a reimbursement requirement, and
generating an interactive surgical plan based on the comparison.

26. The computer-implemented method of any of examples 13-25, further comprising:
creating a virtual 3D model of the one or more patient-specific implants;
converting the virtual 3D model into 3D fabrication data; and
manufacturing at least a portion of the one or more patient-specific implants according to the 3D fabrication data.

27. A system comprising:
one or more processors; and
one or more memories storing instructions that, when executed by the one or more processors, cause the system to perform a process comprising:
   obtaining one or more images of a patient;
   designing a corrected patient pathology based on the one or more images of the patient using a surgical planning and reimbursement platform, wherein the surgical planning and reimbursement platform is programmed to design implants qualifying for at least one medical reimbursement code;
   generating, using the surgical planning and reimbursement platform, an interactive surgical plan that includes a viewable corrected patient pathology for modifying and/or approving the corrected patient pathology; and
   designing, using the surgical planning and reimbursement platform, one or more patient-specific implants for achieving the approved corrected patient pathology and for medical reimbursement under the at least one medical reimbursement code.

28. A non-transitory computer-readable medium storing instructions that, when executed by a computing system, cause the computing system to perform operations comprising:
obtaining one or more images of a patient;
designing a corrected patient pathology based on the one or more images of a patient using a surgical planning and reimbursement platform, wherein the surgical planning and reimbursement platform is programmed to design patient-specific implants qualifying for at least one medical reimbursement code;
generating, using the surgical planning and reimbursement platform, an interactive surgical plan that includes a viewable corrected patient pathology for modifying and/or approving the corrected patient pathology; and
designing, using the surgical planning and reimbursement platform, one or more patient-specific implants for achieving the approved corrected patient pathology and for medical reimbursement under the at least one medical reimbursement code.

29. A computer-implemented method comprising:
obtaining one or more images of a patient;
designing, using a modeling program, a corrected pathology for the patient based on the one or more images using a machine-learning model trained on at least one training set;
obtaining one or more regulatory design constraints for the corrected pathology; and
designing, using the one or more regulatory design constraints, one or more virtual implants configured to achieve the corrected pathology associated with at least one medical reimbursement code.

30. The computer-implemented method of example 29, wherein at least one of the one or more virtual implants represents an orthopedic spinal implant, wherein the one or more regulatory design constraints includes a footprint for the orthopedic spinal implant.

31. The computer-implemented method of any of examples 29-30, further comprising:
selecting reference patient data sets each including one or more patient images and one or more medical reimbursement codes;
training the machine-learning model using the selected reference patient data sets; and
inputting the one or more patient images of the patient into the trained machine-learning model to generate the corrected patient pathology that meets one or more reimbursement parameters.

32. The computer-implemented method of any of examples 29-31, further comprising:
obtaining regulatory approval for implants designed using the machine-learning model.

33. The computer-implemented method of any of examples 29-32, further comprising:
determining one or more primary or second diagnoses of the patient associated with at least one of a diagnosis code, a supply item code, or a procedure code; and
in response to determining the one or more primary or second diagnoses, submitting a payment request associated with one or more patient-specific implants, which were designed based on the one or more virtual implants, under the at least one of the diagnosis code, the supply item code, or the procedure code.

34. A system comprising:
one or more processors; and
one or more memories storing instructions that, when executed by the one or more processors, cause the system to perform a process comprising:
   obtaining one or more images of a patient;
   designing a corrected pathology for the patient based on the one or more images using a machine-learning model trained on at least one training set;
   obtaining one or more regulatory design constraints for the corrected pathology; and
   designing, using the one or more regulatory design constraints one or more virtual implants, one or more patient-specific implants achieving the corrected pathology.

35. A non-transitory computer-readable medium storing instructions that, when executed by a computing system, cause the computing system to perform operations comprising:
obtaining one or more images of a patient;
designing a corrected pathology for the patient based on the one or more images using a machine-learning model trained on at least one training set;
obtaining one or more regulatory design constraints for the corrected pathology; and
designing, using the one or more regulatory design constraints one or more virtual implants, one or more patient-specific implants achieving the corrected pathology.

36. A computer-implemented method comprising:
training a surgical planning and reimbursement platform to design a type of patient-specific implant for a surgical procedure, wherein the surgical planning and reimbursement platform is programmed to design implants qualifying for at least one medical reimbursement; and
inputting patient data into the surgical planning and reimbursement platform to generate a virtual corrected pathology for the patient, and
   design one or more patient-specific implants for the patient to achieve the virtual corrected pathology.

37. The computer-implemented method of example 36, wherein the surgical planning and reimbursement platform includes a trained machine-learning algorithm for designing patient-specific implants.

38. The computer-implemented method of any of examples 36-37, wherein the one or more patient-specific implants are associated with one or more medical codes for reimbursement.

39. The computer-implemented method of any of examples 36-38, wherein the type of patient-specific implant includes an interbody fusion device, an intervertebral artificial disc, and a rod fusion system.

40. The computer-implemented method of any of examples 36-39, wherein the surgical planning and reimbursement platform is programmed to
categorize a pathology of the patient;
select a treatment for the pathology of the patient based on the categorization; and
selecting a type of one or more patient-specific implants for the virtual corrected pathology.

41. The computer-implemented method of any of examples 36-40, further comprising categorizing the pathology of the patient as a spinal curvature deformity, nerve compression, and/or degenerative disc disease.

42. The computer-implemented method of any of examples 36-41, further comprising generating, using the surgical planning and reimbursement platform, a plurality of surgical plans each for implanting a set of implants.

43. The computer-implemented method of any of examples 36-43, wherein at least one of the one or more patient-specific implants is associated with a new technology add-on payment and/or a diagnosis-related group payment.

44. A system comprising:
one or more processors; and
one or more memories storing instructions that, when executed by the one or more processors, cause the system to perform a process comprising:
   training a surgical planning and reimbursement platform to design a type of patient-specific implant for a surgical procedure, wherein the surgical planning and reimbursement platform is programmed to design patient-specific implants qualifying for at least one medical reimbursement; and
   inputting patient data into the surgical planning and reimbursement platform to generate a virtual corrected pathology for the patient, and
   designing one or more patient-specific implants for the patient to achieve the virtual corrected pathology.

45. A non-transitory computer-readable medium storing instructions that, when executed by a computing system, cause the computing system to perform operations comprising:
training a surgical planning and reimbursement platform to design a type of patient-specific implant for a surgical procedure, wherein the surgical planning and reimbursement platform is programmed to design patient-specific implants qualifying for at least one medical reimbursement; and
inputting patient data into the surgical planning and reimbursement platform to generate a virtual corrected pathology for the patient; and
   designing one or more patient-specific implants for the patient to achieve the virtual corrected pathology.

16. A computing system comprising:
one or more processors; and
one or more memories storing instructions that, when executed by the one or more processors, cause the computing system to perform a process of any one of methods in examples 1-45.

17. A non-transitory computer-readable medium storing instructions that, when executed by a computing system, cause the computing system to perform operations of any one of methods in examples 1-45.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, features, systems, devices, materials, methods and techniques described herein may, in some embodiments, be similar to any one or more of the embodiments, features, systems, devices, materials, methods and techniques described in the following:
U.S. application Ser. No. 16/048,167, filed on Jul. 27, 2017, titled “SYSTEMS AND METHODS FOR ASSISTING AND AUGMENTING SURGICAL PROCEDURES;”
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY;”
U.S. application Ser. No. 16/207,116, filed on Dec. 1, 2018, titled “SYSTEMS AND METHODS FOR MULTI-PLANAR ORTHOPEDIC ALIGNMENT;”
U.S. application Ser. No. 16/352,699, filed on Mar. 13, 2019, titled “SYSTEMS AND METHODS FOR ORTHOPEDIC IMPLANT FIXATION;”
U.S. application Ser. No. 16/383,215, filed on Apr. 12, 2019, titled “SYSTEMS AND METHODS FOR ORTHOPEDIC IMPLANT FIXATION;”
U.S. application Ser. No. 16/569,494, filed on Sep. 12, 2019, titled “SYSTEMS AND METHODS FOR ORTHOPEDIC IMPLANTS;” and
U.S. Application No. 62/773,127, filed on Nov. 29, 2018, titled “SYSTEMS AND METHODS FOR ORTHOPEDIC IMPLANTS.”
U.S. Application No. 62/928,909, filed on Oct. 31, 2019, titled “SYSTEMS AND METHODS FOR DESIGNING ORTHOPEDIC IMPLANTS BASED ON TISSUE CHARACTERISTICS;”
U.S. application Ser. No. 16/735,222 (now U.S. Pat. No. 10,902,944), filed Jan. 6, 2020, titled “PATIENT-SPECIFIC MEDICAL PROCEDURES AND DEVICES, AND ASSOCIATED SYSTEMS AND METHODS;”
U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS;”
U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS;”
U.S. application Ser. No. 17/085,564, filed Oct. 30, 2020, titled “SYSTEMS AND METHODS FOR DESIGNING ORTHOPEDIC IMPLANTS BASED ON TISSUE CHARACTERISTICS;”
U.S. application Ser. No. 17/100,396, filed Nov. 20, 2020, titled “PATIENT-SPECIFIC VERTEBRAL IMPLANTS WITH POSITIONING FEATURES;”
U.S. application Ser. No. 17/124,822, filed Dec. 17, 2020, titled “PATIENT-SPECIFIC MEDICAL PROCEDURES AND DEVICES, AND ASSOCIATED SYSTEMS AND METHODS;”
U.S. application Ser. No. 17/518,524, filed Nov. 3, 2021, titled “PATIENT-SPECIFIC ARTHROPLASTY DEVICES AND ASSOCIATED SYSTEMS AND METHODS;”
U.S. application Ser. No. 17/678,874, filed Feb. 23, 2022, (U.S. Pat. No. 11,443,838) titled “NON-FUNGIBLE TOKEN SYSTEMS AND METHODS FOR STORING AND ACCESSING HEALTHCARE DATA;”
U.S. application Ser. No. 18/120,979, filed Feb. 13, 2023, “MULTI-STAGE PATIENT-SPECIFIC SURGICAL PLANS AND SYSTEMS AND METHODS FOR CREATING AND IMPLEMENTING THE SAME;”
U.S. application Ser. No. 18/455,881, filed Aug. 25, 2023, titled “SYSTEMS AND METHODS FOR GENERATING MULTIPLE PATIENT-SPECIFIC SURGICAL PLANS AND MANUFACTURING PATIENT-SPECIFIC IMPLANT;”
U.S. application Ser. No. 17/951,085, filed Sep. 22, 2022, (U.S. Pat. No. 11,806,241) titled “SYSTEM FOR MANUFACTURING AND PRE-OPERATIVE INSPECTING OF PATIENT-SPECIFIC IMPLANTS;”
International Application No. PCT/US2021/012065, filed Jan. 4, 2021, titled “PATIENT-SPECIFIC MEDICAL PROCEDURES AND DEVICES, AND ASSOCIATED SYSTEMS AND METHODS;” and
International Application No. PCT/US23/13744 and U.S. application Ser. No. 18/113,573, filed Feb. 23, 2023, titled “PATIENT-SPECIFIC IMPLANT DESIGN AND MANUFACTURING SYSTEM WITH A DIGITAL FILING CABINET MANAGER.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter. For example, the information, data, and plans can be stored by one or more digital filing cabinets, ledgers, and technology disclosed in International Application No. PCT/US23/13744, filed Feb. 23, 2023, titled “PATIENT-SPECIFIC IMPLANT DESIGN AND MANUFACTURING SYSTEM WITH A DIGITAL FILING CABINET MANAGER.”

The ranges disclosed herein also encompass any and all overlap, sub-ranges, and combinations thereof. Language such as “up to,” “at least,” “greater than,” “less than,” “between,” or the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

What is claimed is:

1. A computer-implemented method for designing patient-specific implant based on a patient-specific interactive surgical plan, the method comprising:
displaying, via a display, the patient-specific interactive surgical plan generated by a surgical planning platform based on one or more images of a patient,
wherein the patient-specific interactive surgical plan includes a user input element for modifying and/or approving the patient-specific interactive surgical plan, wherein the patient-specific interactive surgical plan includes a viewable planned post-operative pathology for the patient,
obtaining reimbursement information for one or more patient-specific implants based on diagnosis data, procedural data, or pathology data of the patient in the patient-specific interactive surgical plan;
assigning at least one medical reimbursement code to the one or more patient-specific implants based on the reimbursement information; and
receiving, via the patient-specific interactive surgical plan, approval of or modification to the planned post-operative pathology, wherein the surgical planning platform is configured to design the one or more patient-specific implants for achieving the approved planned post-operative pathology and for medical reimbursement under the at least one medical reimbursement code.

2. The computer-implemented method of claim 1, wherein the surgical planning platform is configured to
store, via a regulatory and reimbursement manager, a first medical reimbursement code and a second medical reimbursement code in a medical code database;
generate a virtual three-dimensional model representing the patient's anatomy;
manipulate the virtual three-dimensional model to generate a first surgical plan that qualifies for reimbursement under the first medical reimbursement code in the medical code database; and
manipulate the virtual three-dimensional model to generate a second surgical plan that qualifies for reimbursement under the second medical reimbursement code in the medical code database.

3. The computer-implemented method of claim 2, further comprising:
displaying a comparison of the first and second surgical plans, wherein the comparison includes
a reimbursement comparison between the first and second surgical plans; and
a predicted patient outcome comparison between the first and second surgical plans.

4. The computer-implemented method of claim 1, wherein the patient-specific interactive surgical plan includes one or more regulatory indication values associated with a treatment provided by the one or more patient-specific implants.

5. The computer-implemented method of claim 1, further comprising:
displaying a user inputted modification to the patient-specific interactive surgical plan; and
dynamically updating at least one of regulatory approval or reimbursement information, wherein the updating is based on the user inputted modification.

6. The computer-implemented method of claim 1, further comprising displaying one or more user adjustable values,
an anatomical correction based on the one or more user adjustable values, and
reimbursement information associated with treatment for achieving the anatomical correction.

7. The computer-implemented method of claim 1, further comprising:
verifying accuracy of matching of the at least one medical reimbursement code to the one or more patient-specific implants based on at least one predetermined processing rule prior to transmitting a reimbursement request for treatment to a medical imbursement processor;
displaying verification that treatment using the one or more patient-specific implants is approved by a government regulatory agency; and
displaying pricing based on a new technology add-on payment and/or diagnosis-related group payment.

8. The computer-implemented method of claim 1, further comprising:
obtaining one or more pre-operative metrics for pre-determined indications; and
obtaining one or more post-operative metrics to validate a target outcome for the pre-determined indications.

9. The computer-implemented method of claim 1, wherein the obtained reimbursement information includes at least one of treatment type or description of treatment.

10. The computer-implemented method of claim 1, further comprising:
coding one or more patient-specific devices for multiple coded reimbursements,
wherein the multiple coded reimbursements include:
a new technology add-on reimbursement; and
a diagnoses-related groups reimbursement.

11. A system comprising:
one or more processors; and
one or more memories storing instructions that, when executed by the one or more processors, cause the system to perform a process comprising:
displaying, via a display, a patient-specific interactive surgical plan generated by a surgical planning platform based on one or more images of a patient,
wherein the patient-specific interactive surgical plan includes a user input element for modifying and/or approving the patient-specific interactive surgical plan, wherein the patient-specific interactive surgical plan includes a viewable planned post-operative pathology for the patient;
obtaining reimbursement information for one or more patient-specific implants based on diagnosis data, procedural data, or pathology data of the patient in the patient-specific interactive surgical plan;
assigning at least one medical reimbursement code to the one or more patient-specific implants based on the reimbursement information; and
receiving, via the patient-specific interactive surgical plan, approval of or modification to the planned post-operative pathology, wherein the surgical planning platform is configured to design the one or more patient-specific implants for achieving the approved planned post-operative pathology and for medical reimbursement under the at least one medical reimbursement code.

12. The system of claim 11, wherein the process further includes:
storing, via a regulatory and reimbursement manager, a first medical reimbursement code and a second medical reimbursement code in a medical code database;
generating a virtual three-dimensional model representing the patient's anatomy;
manipulating the virtual three-dimensional model to generate a first surgical plan that qualifies for reimbursement under the first medical reimbursement code in the medical code database; and
manipulating the virtual three-dimensional model to generate a second surgical plan that qualifies for reimbursement under the second medical reimbursement code in the medical code database.

13. The system of claim 12, wherein the process further includes:
displaying a comparison of the first and second surgical plans, wherein the comparison includes
a reimbursement comparison between the first and second surgical plans; and
a predicted patient outcome comparison between the first and second surgical plans

14. The system of claim 11, wherein the patient-specific interactive surgical plan includes one or more regulatory indication values associated with a treatment provided by the one or more patient-specific implants.

15. The system of claim 11, wherein the process further includes:
displaying a user inputted modification to the patient-specific interactive surgical plan; and
dynamically updating at least one of regulatory approval or reimbursement information, wherein the updating is based on the user inputted modification.

16. A non-transitory computer-readable medium storing instructions that, when executed by a computing system, cause the computing system to perform operations comprising:
displaying, via a display, a patient-specific interactive surgical plan generated by a surgical planning platform based on one or more images of a patient,
wherein the patient-specific interactive surgical plan includes a user input element for modifying and/or approving the patient-specific interactive surgical plan, wherein the patient-specific interactive surgical plan includes a viewable planned post-operative pathology for the patient,
obtaining reimbursement information for one or more patient-specific implants based on diagnosis data, procedural data, or pathology data of the patient in the patient-specific interactive surgical plan;
assigning at least one medical reimbursement code to the one or more patient-specific implants based on the reimbursement information; and
receiving, via the patient-specific interactive surgical plan, approval of or modification to the planned post-operative pathology, wherein the surgical planning platform is configured to design the one or more patient-specific implants for achieving the approved planned post-operative pathology and for medical reimbursement under the at least one medical reimbursement code.

17. The non-transitory computer-readable medium of claim 16, wherein the operations further include:
storing, via a regulatory and reimbursement manager, a first medical reimbursement code and a second medical reimbursement code in a medical code database;
generating a virtual three-dimensional model representing the patient's anatomy;
manipulating the virtual three-dimensional model to generate a first surgical plan that qualifies for reimbursement under the first medical reimbursement code in the medical code database; and
manipulating the virtual three-dimensional model to generate a second surgical plan that qualifies for reimbursement under the second medical reimbursement code in the medical code database.

18. The non-transitory computer-readable medium of claim 17, wherein the operations further include:
displaying a comparison of the first and second surgical plans, wherein the comparison includes
a reimbursement comparison between the first and second surgical plans; and
a predicted patient outcome comparison between the first and second surgical plans

19. The non-transitory computer-readable medium of claim 16, wherein the patient-specific interactive surgical plan includes one or more regulatory indication values associated with a treatment provided by the one or more patient-specific implants.

20. The non-transitory computer-readable medium of claim 16, wherein the operations further include:
displaying a user inputted modification to the patient-specific interactive surgical plan; and
dynamically updating at least one of regulatory approval or reimbursement information, wherein the updating is based on the user inputted modification.
